(19) United States
(12) Patent Application Publication
(10) Pub. No.: US 2014/0030257 A1
(43) Pub. Date: Jan. 30, 2014

(54) AGTR1 AS A MARKER FOR BEVACIZUMAB COMBINATION THERAPIES

(71) Applicant: Hoffmann-La Roche Inc., Nutley, NJ (US)

(72) Inventors: Juan De La Haba-Rodriguez, Cordoba (ES); Pedro Sanchez-Rovira, Jaen (ES)

(73) Assignee: Hoffmann-La Roche Inc., Nutley, NJ (US)

(21) Appl. No.: 13/911,912

(22) Filed: Jun. 6, 2013

Related U.S. Application Data
(63) Continuation of application No. PCT/EP2011/072026, filed on Dec. 7, 2011.

(30) Foreign Application Priority Data
Dec. 9, 2010 (EP) 10194391.8
Dec. 9, 2010 (EP) 10382332.4

(51) Int. Cl. G01N 33/74; A61K 31/337; C12Q 1/68; A61K 39/395

(52) U.S. Cl. CPC G01N 33/74; A61K 39/39558; A61K 31/337; C12Q 1/6886
USPC 424/133.1; 435/6.11; 435/7.23; 435/6.12

(57) ABSTRACT

The present invention provides methods for assessing the responsiveness or sensitivity of a patient to bevacizumab, compositions comprising bevacizumab and methods of treating patients with bevacizumab.

### RELATED APPLICATIONS

This application is a continuation of International Application No. PCT/EP2011/072026 filed Dec. 7, 2011, the disclosure of which is hereby incorporated by reference in its entirety for all purposes and which claims priority under 35 USC 119 to European Patent Application No. 10382332.4, filed Dec. 9, 2010 and European Patent Application No. 10194391.8, filed Dec. 9, 2010.

### SEQUENCE LISTING

This application contains a Sequence Listing submitted via EFS-Web and hereby incorporated be reference in its entirety. Said ASC11 copy, created on Jun. 3, 2013, is named P4675C1SequenceLisitng.txt, and is 59,755 bytes in size.

### BACKGROUND

Angiogenesis is necessary for cancer development, regulating not only primary tumor size and growth but also impacting invasive and metastatic potential. Accordingly, the mechanisms mediating angiogenic processes have been investigated as potential targets for directed anti-cancer therapies. Early in the study of angiogenic modulators, the VEGF signalling pathway was discovered to preferentially regulate angiogenic activity in multiple cancer types and multiple therapeutics have been developed to modulate this pathway at various points. These therapies include, among others, bevacizumab, sunitinib, sorafenib and vatalanib. Although the use of angiogenic inhibitors in the clinic has shown success, not all patients respond or fail to fully respond to angiogenesis inhibitor therapy. The mechanism(s) underlying such incomplete response is unknown. Therefore, there is an increasing need for the identification of patient subgroups sensitive or responsive to anti-angiogenic cancer therapy.

While a number of angiogenesis inhibitors are known, the most prominent angiogenesis inhibitor is bevacizumab (Avastin®). Bevacizumab is a recombinant humanized monoclonal IgG1 antibody that specifically binds and blocks the biological effects of VEGF. VEGF is a key driver of tumor angiogenesis—an essential process required for tumor growth and metastasis, i.e., the dissemination of the tumor to other parts of the body. Avastin® is approved in Europe for the treatment of the advanced stages of four common types of cancer: colorectal cancer, breast cancer, non-small cell lung cancer (NSCLC) and kidney cancer, which collectively cause over 2.5 million deaths each year. According to the EMEA (European Medicine Agency), Avastin® may be used with other anticancer medicines to treat metastatic cancer of the colon or rectum (large intestine), in combination with chemotherapy that includes a ‘fluoropyrimidine’ (such as 5-fluorouracil) (according to the EMEA, ‘Metastatic’ means that the cancer has spread to other parts of the body); metastatic breast cancer, in combination with paclitaxel or docetaxel; advanced, metastatic or recurrent non-small cell lung cancer that is unresectable (cannot be removed by surgery alone) in patients whose cancer cells are not of the ‘squamous’ type, in combination with chemotherapy that includes a ‘platinum-based’ medicine (‘Advanced’ means that the cancer has started to spread, and ‘recurrent’ means that the cancer has come back after previous treatment); and advanced or metastatic kidney cancer, in combination with interferon alfa-2a. In the USA, Avastin® is further approved for treating glioblastoma multiforme. Over half a million patients have been treated with Avastin® so far, and a comprehensive clinical program with over 450 clinical trials is investigating the further use of Avastin in the treatment of multiple cancer types (including colorectal, breast, non-small cell lung, brain, gastric, ovarian and prostate) in different settings (e.g., advanced or early stage disease). Importantly, Avastin® has shown promising as a co-therapeutic, demonstrating particular efficacy when combined with a broad range of chemotherapies and other anti-cancer treatments. Phase-III studies have been published demonstrating the beneficial effects of combining bevacizumab with standard chemotherapeutic regimens (see, e.g., Kang, J Clin Oncol (2010), 28: 18s (suppl. abstr. LBA4007); Saltz, J Clin Oncol (2008), 26: 2013-2019; Yang, Clin Cancer Res (2008), 14: 5893-5899; Hurwitz, N Eng J Med (2004), 350: 2335-2342). However, as in previous studies of angiogenic inhibitors, some of these phase-III studies have shown that a portion of patients experience incomplete response to the addition of bevacizumab (Avastin®) to their chemotherapeutic regimens.

Accordingly, there is a need for methods of determining those patients that respond or are likely to respond to a therapy comprising angiogenesis inhibitors. Thus, the technical problem underlying the present invention is the provision of means and methods for the identification of (a) patient(s) suffering from or being prone to suffer from a proliferative disease, who may benefit from the treatment with angiogenesis inhibitors.

The technical problem is solved by provision of the embodiments characterized in the claims.

### BRIEF DESCRIPTION OF THE DRAWINGS

FIG. 1: Scheme of one experimental protocol. Pts: Patients. PRF: predictive response factor (biomarker).

### SUMMARY OF THE INVENTION

The present invention provides methods for assessing the responsiveness or sensitivity of a patient to an angiogenesis inhibitor such as a VEGF-binding agent, particularly bevacizumab (Avastin®), either alone or in combination with a chemotherapy regimen, by determining the expression level of angiotensin II type 1 receptor (AGTR1) relative to a control level determined in patients suffering from a proliferative disorder. In particular, the present invention relates to a method for the identification of a patient responsive or sensitive to bevacizumab treatment, said patient being suspected to suffer from or being prone to suffer from a proliferative disorder, wherein said method comprises the step of determining the expression level of angiotensin II type 1 receptor (AGTR1) in a biological sample of said patient, wherein an increased expression level of AGTR1 compared to a control level determined in patients suffering from cancer is indicative for said patient to be responsive or sensitive to bevacizumab treatment.

The present invention also provides a composition comprising an angiogenesis inhibitor such as a VEGF-binding agent, particularly bevacizumab (Avastin®) for use in the treatment of a proliferative disorder in a patient identified by the method provided and disclosed herein, the use of an angiogenesis inhibitor such as a VEGF-binding agent, particularly bevacizumab (Avastin®) for the preparation of a pharmaceutical composition for the treatment of a proliferative disorder in a patient identified by the method provided and disclosed herein, as well as for methods for the treatment of a proliferative disorder comprising administering an effective amount of an angiogenesis inhibitor such as a VEGF-binding agent, particularly bevacizumab (Avastin®) to a subject identified by the method provided and disclosed herein.

The present invention, therefore, provides a method for the identification of a patient responsive or sensitive to bevacizumab treatment, said patient being suspected to suffer from or being prone to suffer from a proliferative disorder, wherein said method comprises the step of determining the expression level of angiotensin II type 1 receptor (AGTR1) in a biological sample of said patient,

wherein an increased expression level of AGTR1 compared to a control level determined in patients suffering from the same proliferative disorder is indicative for said patient to be responsive or sensitive to bevacizumab treatment.

In context with the present invention, biological samples include biopsies (e.g., core biopsies), tissue resections, and body fluids, e.g., blood samples comprising cancer/tumor cells, of a patient suffering from, being suspected to suffer from, being prone to suffer from or diagnosed with a proliferative disease as described herein.

In context with the present invention, a biomarker of a proliferative disorder, particularly breast cancer, has been identified that correlates with sensitivity or responsiveness of a patient to angiogenesis inhibitors such as a VEGF-binding agent, e.g., bevacizumab (Avastin®), either alone or, in particular, in combination with chemotherapeutic regimens. In certain aspects, the invention relates to the tumor specific expression level of AGTR1 determined relative to controls established in patients suffering from breast cancer to identify patients which are sensitive or responsive to the administration of bevacizumab (Avastin®), either alone or in combination with a chemotherapeutic regimen.

The present invention, therefore, provides a method for the identification of a patient responsive or sensitive to bevacizumab treatment, said patient being suspected to suffer from or being prone to suffer from a proliferative disorder, wherein said method comprises the step of determining the expression level of angiotensin II type 1 receptor (AGTR1) in a biological sample of said patient;

wherein an increased expression level of AGTR1 compared to a control level determined in patients suffering from the same proliferative disorder is indicative for said patient to be responsive or sensitive to bevacizumab treatment,
wherein said bevacizumab treatment is comprised in a combination therapy including a chemotherapy.

The present invention also relates to a method for improving the treatment effect of bevacizumab treatment alone or in combination with a chemotherapy regimen of a patient suffering from a proliferative disorder by administering bevacizumab alone or in combination with to the chemotherapy regimen, said method comprising:
(a) determining the expression level of AGTR1 in a biological sample of said patient; and
(b) administering bevacizumab alone or in combination with a chemotherapy regimen to the patient having an increased expression level of AGTR1 relative to control expression levels determined in patients diagnosed with the same proliferative disorder.

Hence, the present invention relates to a method for improving the treatment effect of a bevacizumab treatment alone or in addition to a chemotherapy regimen of patients suffering or being pone to suffer from a proliferative disease as described herein, in particular breast cancer such as locally advanced, recurrent or metastatic HER2 negative breast cancer, by administering bevacizumab alone or in combination with a chemotherapy regimen to patients in which an increased expression level of AGTR1 has been determined as described and exemplified herein.

Accordingly, the present invention solves the technical problem in that it was surprisingly found that the expression level of AGTR1 in a given patient, relative to a control level determined in patients diagnosed with a proliferative disorder, correlates with treatment effect in those patients using an angiogenesis inhibitor such as a VEGF-binding agent, e.g., bevacizumab. According to the present invention, this particularly applies wherein the bevacizumab treatment is conducted in form of a combination therapy or comprised in a combination therapy.

These and other embodiments of the invention are further described in the detailed description that follows.

### DETAILED DESCRIPTION OF THE INVENTION

As already mentioned, bevacizumab (Avastin®) is approved in Europe for the treatment of the advanced stages of four common types of cancer: breast cancer, colorectal cancer (CRC), non-small cell lung cancer (NSCLC) and kidney cancer (e.g., renal cell carcinoma, RCC). Also, bevacizumab (Avastin®) has been shown to be effective in treatment of brain cancer, particularly glioblastoma multiforme (GBM) and ovarian cancer. Further potential uses of bevacizumab (Avastin®) are treatment of prostate cancer (particularly castrate-resistant prostate cancer), liver cancer (particularly non-metastatic unresectable liver cancer), melanoma, bladder cancer, cervical carcinoma, gastric cancer, carcinoid and pancreatic cancer (metastatic or unresectable locally advanced pancreatic cancer).

Accordingly, the term “proliferative disorder” as used herein may refer to breast cancer (e.g., locally advanced, recurrent or metastatic HER2 negative breast cancer), colorectal cancer (CRC), non-small cell lung cancer (NSCLC), kidney cancer (e.g., renal cell carcinoma, RCC), brain cancer, particularly glioblastoma multiforme (GBM), ovarian cancer, prostate cancer (particularly castrate-resistant prostate cancer), liver cancer (particularly non-metastatic unresectable liver cancer), melanoma, bladder cancer, cervical carcinoma, gastric cancer, carcinoid and pancreatic cancer (metastatic or unresectable locally advanced pancreatic cancer). Preferably, in context with the present invention, the proliferative disorder is breast cancer, in particular locally advanced, recurrent or metastatic HER2 negative breast cancer.

In context with the present invention, it was particularly shown that AGTR1 overexpression is correlated with a high percentage of pathological complete responders (pCR) in breast cancer patients who were treated with bevacizumab (Avastin®), particularly in combination with chemotherapeutic regimens. In context with the present invention, the expression level of AGTR1 was surprisingly identified as a biomarker (predictive response factor, PRF) for a high percentage of pCR in breast cancer patients in response to treatment with bevacizumab (Avastin®), particularly in combination with chemotherapeutic regimens. Specifically, breast cancer patients exhibiting a response or sensitivity to treatment with bevacizumab (Avastin®) were identified to have increased expression of AGTR1 relative to a control level established in biological samples of patients suffering from or diagnosed with breast cancer. The terms “biomarker” and “predictive response factor (PRF)” can be used interchangeably and refer to the expression level of AGTR1 as described and defined herein.

Accordingly, the present invention relates to a method for the identification of a patient responsive or sensitive to bevacizumab treatment, said patient being suspected to suffer from or being prone to suffer from breast cancer, wherein said method comprises the step of determining the expression level of angiotensin II type 1 receptor (AGTR1) in a biological sample of said patient;

wherein an increased expression level of AGTR1 compared to a control level determined in patients suffering from beast cancer is indicative for said patient to be responsive or sensitive to bevacizumab treatment,
wherein said bevacizumab treatment is comprised in a combination therapy including a chemotherapy as described herein.

In the context of the present invention, “AGTR1” refers to the angiotensin II type 1 receptor (Peach, Physiol Rev (1977), 57: 313-370;), also known as AT1, and exemplified by the amino acid sequence SEQ ID NO: 2 (see Swiss Prot Accession No. P30556.1); the mRNA/cDNA of AGTR1 as used herein is exemplified by the nucleotide sequence shown in SEQ ID NO: 1 (see GenBank Accession No. AY221090.1). The nucleotide sequences disclosed herein are shown as the complete coding sequences of the indicated proteins using the nucleotide bases adenine (a), guanine (g), cytosine (c) and thymine (t). The person skilled in the art readily knows that within RNA, thymine is replaced by uracil (u). As used herein, “AGTR1” also encompasses homologs, variants and isoforms of AGTR1, so long as said homologs, variants and isoforms are specifically recognized or detectable by agents suitable to determine expression of AGTR1, such as anti-AGTR1 binding agents (e.g., antibodies) binding to the AGTR1 protein or nucleic acid molecules hybridizing to the nucleotide sequence of AGTR1 cDNA or mRNA (e.g., probes or primers). Such agents are described herein and/or known in the art. Also, methods of determining the expression of AGTR1 protein or mRNA using such agents are described herein and/or known in the art. The term “AGTR1” further encompasses proteins or nucleotide sequences having at least 70%, at least 75%, at least 80%, at least 85%, at least 90% or at least 95% identity to the amino acid sequence of SEQ ID NO: 2 or the nucleotide sequence of SEQ ID NO: 1, respectively, or to the sequence of one or more of a AGTR1 homologue, variant and isoform, including splice isoforms, as well as fragments of the sequences, provided that the variant proteins (including isoforms), homologous proteins and/or fragments are recognized or detectable by agents suitable to determine expression of AGTR1 protein or AGTR1 mRNA.

In order to determine whether an amino acid or nucleic acid sequence has a certain degree of identity to an amino acid or nucleic acid sequence as herein described, the skilled person can use means and methods well known in the art, e.g. alignments, either manually or by using computer programs known in the art or described herein.

In accordance with the present invention, the terms “identical” or “identity” in the context of two or more or amino acid or nucleic acid sequences, refers to two or more sequences or subsequences that are the same, or that have a specified percentage of amino acid residues or nucleotides that are the same (e.g., 70% or 75% identity, preferably, 80-95% identity, more preferably at least 95% identity with the amino acid sequences of, e.g., SEQ ID NO: 1 or SEQ ID NO: 2), when compared and aligned for maximum correspondence over a window of comparison, or over a designated region as measured using a sequence comparison algorithm as known in the art, or by manual alignment and visual inspection. Sequences having, for example, 70% to 95% or greater sequence identity are considered to be substantially identical. Such a definition also applies to the complement of a test sequence. Preferably the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson, Nucl Acids Res (1994), 2: 4673-4680) or FASTDB (Brutlag, Comp App Biosci (1990), 6: 237-245), as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST (Basic Local Alignment Search Tool) and BLAST 2.0 algorithms (Altschul, Nucl Acids Res (1997), 25: 3389-3402; Altschul, J Mol Evol (1997), 36: 290-300; Altschul, J Mol Biol (1990), 215:403-410). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff (1989) PNAS 89:10915) uses alignments (B) of 50, expectation (E) of 10, M=5, N=4, and a comparison of both strands.

BLAST algorithms, as discussed above, produce alignments of both amino and nucleotide sequences to determine sequence similarity. Because of the local nature of the alignments, BLAST is especially useful in determining exact matches or in identifying similar sequences. The fundamental unit of BLAST algorithm output is the High-scoring Segment Pair (HSP). An HSP consists of two sequence fragments of arbitrary but equal lengths whose alignment is locally maximal and for which the alignment score meets or exceeds a threshold or cut-off score set by the user. The BLAST approach is to look for HSPs between a query sequence and a database sequence, to evaluate the statistical significance of any matches found, and to report only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

Analogous computer techniques using BLAST may be used to search for identical or related molecules in protein or nucleotide databases such as GenBank or EMBL. This analysis is much faster than multiple membrane-based hybridizations. In addition, the sensitivity of the computer search can be modified to determine whether any particular match is categorized as exact or similar. The basis of the search is the product score which is defined as:

$$\frac{\% sequence identity \times \% maximum BLAST score}{100}$$

and takes into account both the degree of similarity between two sequences and the length of the sequence match. For example, with a product score of 40, the match will be exact within a 1-2% error; and at 70, the match will be exact. Similar molecules are usually identified by selecting those which show product scores between 15 and 40, although lower scores may identify related molecules. Another example for a program capable of generating sequence alignments is the CLUSTALW computer program (Thompson, Nucl Acids Res (1994), 2: 4673-4680) or FASTDB (Brutlag, Comp App Biosci (1990), 6: 237-245), as is known in the art.

In accordance with the present invention, it was surprisingly discovered that a greater bevacizumab treatment effect was associated with higher AGTR1 expression. Specifically, relatively higher AGTR1 expression was associated with improved pathological complete response (pCR) in patients receiving bevacizumab in addition to the chemotherapeutic regimen.

The expression level of AGTR1 or a variant, homologue, truncation or fragment thereof may be assessed by any method known in the art suitable for determination of specific protein or mRNA levels in a biological patient sample. In one embodiment, the expression level of AGTR1 protein is determined by an immunohistochemical (IHC) method employing antibodies specific for AGTR1 as known in the art and as also described and exemplified herein. Further suitable methods include, but are not limited to, ICC (immunocytochemistry), RIA (Radio Immuno Assay), sandwich (immunometric assay), Western blot, IRMA (Immune Radioimmunometric Assay), EIA (Enzyme Immuno Assay), ELISA (Enzyme Linked Immuno Assay), FIA (Fluorescent Immuno Assay), CLIA (Chemioluminescent Immune Assay). Such methods are well known and routinely implemented in the art and corresponding antibodies and/or kits are readily available and/or they can be generated by routine methods known in the art. For example, commercially available antibodies specific for AGTR1 as described and defined herein can be obtained from, e.g., Millipore™, USA (for example anti-AGTR1 antibody AB15552) or from Santa Cruz Biotechnology, USA (antibody sc-1173). Preferably, the expression levels of the marker/indicator proteins of the invention are assessed using the reagents and/or protocol recommendations of the antibody or kit manufacturer. The skilled person will also be aware of further means and methods for determining the expression level of AGTR1 protein by suitable methods such as IHC. For determining the expression level of AGTR1 mRNA, methods known in the art can be applied. Such methods include, but are not limited to, PCR, qPCR, RT-PCR, qRT-PCR, RT-qPCR, sequencing (optionally subsequent to a PCR qPCR, RT-PCR, qRT-PCR or RT-qPCR), Light Cycler®, TaqMan° Platform and Assays or quantigene assay (Zhou, Anal Biochem (2000), 282: 46-53), an in situ hybridization method such as fluorescent in situ hybridization (FISH), chromogenic in situ hybridization (CISH) or silver in situ hybridization (SISH), Northern blot, dot blot, microarrays, or next generation sequencing (VanGuilder, Biotechniques (2008), 44(5): 619-26; Elvidge, Pharmacogenomics (2006), 7: 123-134; Metzker, Nat Rev Genet (2010), 11: 31-46; Kafatos, NAR (1979), 7: 1541-1552). In one embodiment of the present invention, the expression level of AGTR1 mRNA is determined by using a PCR such as RT-PCR as known in the art and as also described and exemplified herein. Therefore, the expression level of AGTR1 and/or other markers/indicators as known in the art can be routinely and reproducibly determined by a person skilled in the art without undue burden. However, to ensure accurate and reproducible results, the invention also encompasses the testing of patient samples in a specialized laboratory that can ensure the validation of testing procedures.

Preferably, the expression level of AGTR1 is assessed in a biological sample that contains or is suspected to contain cancer cells and is determined in a tumor-specific manner. The sample may comprise both cell types, i.e., tumor cells, and non-cancerous cells, e.g., non-malignant cells. In some aspects, determination of the expression level of AGTR1 relates to the determination of the expression levels of exclusively cancer cells as opposed to other cell types, e.g., non-cancerous/non-malignant cells, that may be present in the sample. In other aspects, determination of the expression level of AGTR1 relates to the determination of expression levels of cancer cells as well as any other cell-type, that may be present in the sample. The skilled artisan can readily discern cancer cells from non-cancerous cells. The sample may be a tissue biopsy, e.g. a core biopsy, or a tissue resection of a patient suffering from, being suspected to suffer from, being prone to suffer from or diagnosed with a proliferative disease as described herein, in particular, breast cancer, more particularly locally advanced, recurrent or metastatic HER2 negative breast cancer. The sample may also be a resection or biopsy (e.g., core biopsy) of a metastatic lesion obtained from a patient suffering from, being suspected to suffer from, being prone to suffer from or diagnosed with a proliferative disorder as described herein. In context with the present invention, biological samples include biopsies (e.g., core biopsies), tissue resections, and body fluids, e.g., blood samples comprising cancer/tumor cells, as well known in the art. Preferably, when the proliferative disorder referred to in the methods provided and described herein is breast cancer, the sample is a sample of breast tissue. In this context, the sample may also be a sample of a known or suspected metastatic breast cancer lesion or section, or a blood sample, e.g., a peripheral blood sample, known or suspected to comprise circulating cancer cells, e.g., breast cancer cells. The analysis of the sample according to the methods of the invention may be manual, as performed by the skilled artisan, as is known in the art, or may be automated using commercially available software designed for the processing and analysis of pathology images, e.g., for analysis in tissue biopsies or resections (e.g., MIRAX SCAN, Carl Zeiss AG, Jena, Germany). In context with the means and methods described and provided herein, the sample may have been collected before or after the patient has been treated with bevacizumab and/or any chemotherapy as described herein. In one embodiment, the sample has been collected before the patient has been treated with bevacizumab and/or any chemotherapy as described herein.

In accordance with the means and methods described herein in context with the present invention, the patient being suspected to suffer from or being prone to suffer from a proliferative disorder as described herein may be mammal. Preferably, the patient is human, particularly a female human.

In the context of the present invention, bevacizumab treatment may mean that bevacizumab is to be administered alone or that is comprised in a combination therapy, i.e. that bevacizumab is to be administered in addition to or as a co-therapy or co-treatment with one or more other therapeutic agents, e.g., chemotherapeutic agents administered as part of standard chemotherapy regimen as known in the art. Examples of such chemotherapeutic agents include, but are not limited to, docetaxel, cyclophosphamide, epirubicin, doxorubicin, fluorouracil, xeloda, fluoropyrimidine, cisplatin, anthracycline/taxane, anti-metabolite agent, anti-hormonal compound, tyrosine kinase inhibitor, raf inhibitor, ras inhibitor, dual tyrosine kinase inhibitor, taxane, 5-fluorouracil, leucovorin, irinotecan, gemcitabine-erlotinib, capecitabine, mTOR-inhibitors and platinum-based chemotherapeutic agents, such as paclitaxel/taxol, carboplatin, cisplatin and oxaliplatin.

Accordingly, the present invention relates to a method for the identification of a patient responsive or sensitive to bevacizumab treatment, said patient being suspected to suffer from or being prone to suffer from breast cancer, wherein said method comprises the step of determining the expression level of angiotensin II type 1 receptor (AGTR1) in a biological sample of said patient,

wherein an increased expression level of AGTR1 compared to a control level determined in patients suffering from breast cancer is indicative for said patient to be responsive or sensitive to bevacizumab treatment, and
wherein said bevacizumab treatment is comprised in a combination therapy including a chemotherapy such as docetaxel therapy.

Furthermore, in context of the means and methods described and provided herein, bevacizumab treatment may be comprised in a combination therapy which is a combination of bevacizumab treatment and chemotherapy. As mentioned, the chemotherapy may be a therapy with one, two or more selected from the group consisting of docetaxel, cyclophosphamide, epirubicin, doxorubicin, fluorouracil, xeloda, fluoropyrimidine, cisplatin, anthracycline/taxane, anti-metabolite agent, anti-hormonal compound, tyrosine kinase inhibitor, raf inhibitor, ras inhibitor, dual tyrosine kinase inhibitor, taxane, and adjuvant (anti-) hormone drugs, 5-fluorouracil, leucovorin, irinotecan, gemcitabine-erlotinib, capecitabine and platinum-based chemotherapeutic agents, such as paclitaxel/taxol, carboplatin, cisplatin and oxaliplatin. In one embodiment, the chemotherapy is docetaxel therapy. Furthermore, a chemotherapy in context with the methods of the present invention may be a combination therapy selected from the group consisting of a combination of docetaxel and cyclophosphamide, a combination of fluoropyrimidine and cisplatin, a combination of docetaxel and paclitaxel/taxol, a combination of epirubicin and cyclophosphamide, a combination of doxorubicin and cyclophosphamide, a combination of epirubicin and fluorouracil, and a combination of doxorubicin and fluorouracil. In one embodiment, the chemotherapy is a combination of doxorubicin therapy and cyclophosphamide therapy. In another embodiment, the chemotherapy is a combination of docetaxel therapy, doxorubicin therapy and cyclophosphamide therapy.

Accordingly, the present invention relates to a method for the identification of a patient responsive or sensitive to bevacizumab treatment, said patient being suspected to suffer from or being prone to suffer from breast cancer, wherein said method comprises the step of determining the expression level of angiotensin II type 1 receptor (AGTR1) in a biological sample of said patient,

wherein an increased expression level of AGTR1 compared to a control level determined in patients suffering from breast cancer is indicative for said patient to be responsive or sensitive to bevacizumab treatment, and
wherein said bevacizumab treatment is comprised in a combination therapy including a chemotherapy such as docetaxel therapy which is conducted after treatment of said patient with a combination chemotherapy such as doxorubicin/cyclophosphamide therapy.

Generally, in context of the means and methods described and provided herein, the bevacizumab treatment, either alone or in combination with other therapeutics such as chemotherapeutics, may be conducted or applied before or after a chemotherapy. For example, a patient being suspected to suffer from or being prone to suffer from a proliferative disorder as described herein may be treated with a combination of bevacizumab and a chemotherapeutic (e.g., docetaxel) subsequently to chemotherapy without bevacizumab (e.g., treatment with doxorubicin and/or cyclophosphamide) as described herein. Also, a patient being suspected to suffer from or being prone to suffer from a proliferative disorder as described herein may be treated with a combination of bevacizumab and a chemotherapeutic (e.g., docetaxel) prior to chemotherapy without bevacizumab (e.g., treatment with doxorubicin and/or cyclophosphamide) as described herein. In one embodiment, the patient is first treated with a chemotherapy without bevacizumab (e.g., treatment with a combination of doxorubicin and cyclophosphamide) and then treated with bevacizumab alone or with a combination of bevacizumab and docetaxel. In accordance with the present invention, expression level of AGTR1 may be determined before or after the patient is treated with bevacizumab and/or chemotherapy. In one embodiment, the AGTR1 expression level is determined before the patient is treated with bevacizumab and/or chemotherapy.

Generally, administration of bevacizumab or chemotherapeutics as described herein may be applied by parenteral, oral, intravenous, intraperitoneal, subcutaneous, intramuscular, topical, intradermal, intranasal or intrabronchial (for example as effected by inhalation) route. Common modes of administration include parenteral administration as a bolus dose or as an infusion over a set period of time, e.g., administration of the total daily dose over 10 min, 20 min, 30 min, 40 min, 50 min, 60 min, 75 min, 90 min, 105 min, 120 min, 3 hr, 4 hr, 5 h. or 6 hr. For example, 2.5 mg/kg of body weight to 15 mg/kg of body weight bevacizumab (Avastin®) can be administered every week, every 2 weeks or every 3 weeks, depending on the type of cancer being treated. Examples of dosages include 2.5 mg/kg of body weight, 5 mg/kg of body weight, 7.5 mg/kg of body weight, 10 mg/kg of body weight and 15 mg/kg of body weight given every week, every 2 weeks or every 3 weeks. Further examples of dosages are 5 mg/kg of body weight every 2 weeks, 10 mg/kg every 2 weeks, 7.5 mg/kg of body weight every 3 weeks and 15 mg/kg of body weight every 3 weeks. In the context of the herein described invention, low dose bevacizumab includes, for example, dosages of 2.5 mg/kg of body weight every week, 5 mg/kg of body weight every 2 weeks and 7.5 mg/kg of body weight every 3 weeks. In the context of the herein described invention, high dose bevacizumab includes, for example, dosages of 5 mg/kg of body weight every week, 10 mg/kg of body weight every 2 weeks and 15 mg/kg of body weight every 3 weeks. For the treatment of breast cancer, in particular locally advanced, recurrent or metastatic HER2 negative breast cancer, dosages include low dose bevacizumab, in particular 7.5 mg/kg every 3 weeks, and high dose bevacizumab, in particular 15 mg/kg of body weight given once every 3 weeks. The skilled person will recognize that further modes of administration of bevacizumab are encompassed by the invention as determined by the specific patient and chemotherapy regimen, and that the specific mode of administration and therapeutic dosage are best determined by the treating physician according to methods known in the art.

Also, in context with the present invention, the patient may be co-treated with one or more additional anti-cancer therapies, e.g., radiation therapy.

Generally, in context with the means and methods described and provided herein, the therapy with bevacizumab and/or any combination therapy comprising further therapeutics such as chemotherapeutics may be neoadjuvant or adjuvant, preferably it is neoadjuvant.

In context with the present invention, the expression level of AGTR1 may be determined before (“at baseline”) or after (e.g., at surgery) the patient has been treated with bevacizumab and/or any combination therapy comprising further therapeutics such as chemotherapeutics. In one embodiment, the expression level of AGTR1 is determined before the patient has been treated with bevacizumab and/or any combination therapy comprising further therapeutics such as chemotherapeutics, i.e. the AGTR1 expression level is determined at baseline. Also, the AGTR1 expression level may be determined before or after neoadjuvant or adjuvant therapy as described herein. In one embodiment, the AGTR1 expression level is determined before neoadjuvant therapy.

As described and exemplified herein, the addition of bevacizumab to docetaxel-based chemotherapeutic regimen subsequent to treatment with doxorubicin/cyclophosphamide-based chemotherapy effected an increase in the percentage of pathological complete responders (pCR) in breast cancer patients having increased expression of AGTR1 in tumor samples relative to control levels established in similarly situated patients.

The present invention also relates to a composition or kit comprising oligonucleotides or polypeptides suitable for the determination of the expression level of AGTR1. As detailed herein, oligonucleotides such as DNA, RNA or mixtures of DNA and RNA probes may be of use in detecting mRNA levels of AGTR1, while polypeptides (e.g., antibodies) may be of use in directly detecting protein levels of the marker/indicator proteins via specific protein-protein interaction. In preferred aspects of the invention, the polypeptides encompassed as probes for the expression levels of AGTR1, and included in the kits or compositions described herein, are antibodies specific for AGTR1, or specific for homologues, variants and/or truncations thereof.

Accordingly, a further embodiment of the present invention provides a kit useful for carrying out the methods herein described and provided, comprising oligonucleotides or polypeptides capable of determining the expression level of AGTR1. Preferably, the oligonucleotides comprise primers and/or probes specific for the mRNA encoding AGTR1 as defined and described herein, and the polypeptides comprise proteins capable of specific interaction with AGTR1, e.g., marker/indicator specific antibodies or antibody fragments.

Also provided herein are compositions comprising bevacizumab for use in the treatment of a proliferative disorder as described herein in a patient identified by a method as described and provided herein. The compositions may be pharmaceutical composition additionally comprising a pharmaceutically acceptable carrier and/or diluent as known in the art. Generally, examples of suitable pharmaceutical carriers are well known in the art and include phosphate buffered saline solutions, water, emulsions, such as oil/water emulsions, various types of wetting agents, non-aqueous and aqueous solutions, sterile solutions etc. Compositions comprising such carriers can be formulated by well known conventional methods. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Also, the present invention relates to the use of bevacizumab for the preparation of a medicament or a pharmaceutical composition for the treatment of a proliferative disorder as described herein in a patient identified by the method as described and provided herein. Furthermore, herein provided are methods for the treatment of a proliferative disorder as described herein in a patient identified by the methods as described and provided herein.

As documented in the appended examples, the present invention solves the identified technical problem in that it could surprisingly be shown that the expression level of AGTR1 in a given patient, relative to a control level determined in patients diagnosed with a proliferative disorder (e.g., breast cancer), correlate with treatment effect in patients administered with bevacizumab, particularly in combination with a chemotherapy regimen. Particularly, in context with the present invention, it has been shown that patients having a proliferative disorder, particularly breast cancer patients, and having an increased expression level of AGTR1 are more likely to be pathological complete responders (pCR) after being treated with bevacizumab, particularly in combination with a chemotherapy regimen. As exemplified herein, this particularly applies for HER2 negative patients diagnosed with locally advanced breast cancer. Accordingly, as has been shown in accordance with the present invention, patients being suspected to suffer from or being prone to suffer from a proliferative disorder as described herein and having an increased expression level of AGTR1 are considered to be responsive to or sensitive to bevacizumab treatment, either alone or in combination with a chemotherapy regimen as described herein.

The phrase “responsive to” in the context of the present invention indicates that a patient suffering from, being suspected to suffer or being prone to suffer from, or diagnosed with a proliferative disorder as described herein, shows a response to bevacizumab treatment, particularly comprised in a combination therapy including a chemotherapy regimen. The skilled person will readily be in a position to determine whether a person treated with bevacizumab according to the methods of the invention shows a response. For example, a response may be reflected by decreased suffering from the proliferative disorder, such as a diminished and/or halted tumor growth, reduction of the size of a tumor, and/or amelioration of one or more symptoms of the proliferative disorder.

The phrase “sensitive to” in the context of the present invention indicates that a patient suffering from, being suspected to suffer or being prone to suffer from, or diagnosed, with a proliferative disorder as described herein, shows in some way a positive reaction to treatment with bevacizumab, particularly in combination with a chemotherapy regimen. For example, the patient may experience less suffering associated with the disorder, though no reduction in tumor growth or metastatic indicator may be measured, and/or the reaction of the patient to the bevacizumab, either alone or in combination with the chemotherapy regimen, may be only of a transient nature, i.e. the growth of (a) tumor and/or (a) metastasis(es) may only be temporarily reduced or halted.

The phrase “a patient suffering from” in accordance with the invention refers to a patient showing clinical signs of a proliferative disorder as described herein, in particular, breast cancer. The phrase “being susceptible to” or “being prone to,” in the context of the proliferative disorder refers to an indication disease in a patient based on, e.g., a possible genetic predisposition, a pre- or eventual exposure to hazardous and/or carcinogenic compounds, or exposure to carcinogenic physical hazards, such as radiation.

The phrase “treatment effect” as used herein particularly relates to the percentage of pathological complete responders (pCR) as it is known in the art.

The terms “administration” or “administering” as used herein mean the administration of an angiogenesis inhibitor such as a VEGF-binding agent, e.g., bevacizumab (Avastin®), and/or a pharmaceutical composition/treatment regimen comprising an angiogenesis inhibitor such as a VEGF-binding agent, e.g., bevacizumab (Avastin®), to a patient in need of such treatment or medical intervention by any suitable means known in the art for administration of a therapeutic antibody. Non-limiting routes of administration include oral, intravenous, intraperitoneal, subcutaneous, intramuscular, topical, intradermal, intranasal or intrabronchial (for example as effected by inhalation) route. Particularly preferred in context of this invention is parenteral administration, e.g., intravenous administration.

The term “antibody” is herein used in the broadest sense and includes, but is not limited to, monoclonal and polyclonal antibodies, multispecific antibodies (e.g., bispecific antibodies), chimeric antibodies, CDR grafted antibodies, humanized antibodies, camelized antibodies, single chain antibodies and antibody fragments and fragment constructs, e.g., F(ab′)₂ fragments, Fab-fragments, Fv-fragments, single chain Fv-fragments (scFvs), bispecific scFvs, diabodies, single domain antibodies (dAbs) and minibodies, which exhibit the desired biological activity, in particular, specific binding to one or more of AGTR1 or to homologues, variants, fragments and/or isoforms thereof as described herein.

In addition to the examples provided above, in context of the present invention, a chemotherapeutic agent includes any active agent that can provide an anticancer therapeutic effect and may be a chemical agent or a biological agent, in particular, that are capable of interfering with cancer or tumor cells. Such active agents may be those that act as anti-neoplastic (chemotoxic or chemostatic) agents which inhibit or prevent the development, maturation or proliferation of malignant cells. Non-limiting examples of chemotherapeutic agents include alkylating agents such as nitrogen mustards (e.g., mechlorethamine, cyclophosphamide, ifosfamide, melphalan and chlorambucil), nitrosoureas (e.g., carmustine (BCNU), lomustine (CCNU), and semustine (methyl-CCNU)), ethylenimines/methylmelamines (e.g., thriethylenemelamine (TEM), triethylene, thiophosphoramide (thiotepa), hexamethylmelamine (HMM, altretamine)), alkyl sulfonates (e.g., busulfan), and triazines (e.g., dacarbazine (DTIC)); antimetabolites such as folic acid analogs (e.g., methotrexate, trimetrexate), pyrimidine analogs (e.g., 5-fluorouracil, fluorodeoxyuridine, gemcitabine, cytosine arabinoside (AraC, cytarabine), 5-azacytidine, 2,2′-difluorodeoxycytidine, and pyrimidine analog prodrugs, e.g., capecitabine), purine analogs (e.g., 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA)); antimitotic drugs developed from natural products (e.g., paclitaxel, vinca alkaloids (e.g., vinblastine (VLB), vincristine, and vinorelbine), taxotere, estramustine, and estramustine phosphate), epipodophylotoxins (.e.g., etoposide, teniposide), antibiotics (.e.g, actimomycin D, daunomycin (rubidomycin), doxorubicin, mitoxantrone, idarubicin, bleomycins, plicamycin (mithramycin), mitomycinC, actinomycin), enzymes (e.g., L-asparaginase), and biological response modifiers (e.g., interferon-alpha, IL-2, G-CSF, GM-CSF); miscellaneous agents including platinum coordination complexes (e.g., cisplatin, carboplatin), anthracenediones (e.g., mitoxantrone), substituted urea (i.e., hydroxyurea), methylhydrazine derivatives (e.g., N-methylhydrazine (MIH), procarbazine), adrenocortical suppressants (e.g., mitotane (o,p′-DDD), aminoglutethimide); hormones and antagonists including adrenocorticosteroid antagonists (.e.g, prednisone and equivalents, dexamethasone, aminoglutethimide), progestins (e.g., hydroxyprogesterone caproate, medroxyprogesterone acetate, megestrol acetate), estrogens (e.g., diethylstilbestrol, ethinyl estradiol and equivalents thereof); antiestrogens (e.g., tamoxifen), androgens (e.g., testosterone propionate, fluoxymesterone and equivalents thereof), antiandrogens (e.g., flutamide, gonadotropin-releasing hormone analogs, leuprolide) and non-steroidal antiandrogens (e.g., flutamide).

As known in the art, the term “polypeptide” relates to a peptide, a protein, an oligopeptide or a polypeptide which encompasses amino acid chains of a given length, wherein the amino acid residues are linked by covalent peptide bonds. However, peptidomimetics of such proteins/polypeptides are also encompassed by the invention wherein amino acid(s) and/or peptide bond(s) have been replaced by functional analogs, e.g., an amino acid residue other than one of the 20 gene-encoded amino acids, e.g., selenocysteine. Peptides, oligopeptides and proteins may be termed polypeptides. The terms polypeptide and protein are used interchangeably herein. The term polypeptide also refers to, and does not exclude, modifications of the polypeptide, e.g., glycosylation, acetylation, phosphorylation and the like. Such modifications are well described in basic texts and in more detailed monographs, as well as in a voluminous research literature.

The terms “treating” and “treatment” as used herein refer to remediation of, improvement of, lessening of the severity of, or reduction in the time course of the disease or any parameter or symptom thereof.

As mentioned above, the increased expression of AGTR1 according to the present invention may be reflected in the determination of the expression level of AGTR1 protein or mRNA. Generally, in context of the means and methods described herein, the expression level of AGTR1 protein may be considered as “increased” or “overexpressed” if AGTR1 protein can be detected in a biological patient sample (e.g., a breast tissue core biopsy) by a detection method such as IHC, while in the control sample, AGTR1 protein cannot be measured using the same detection method. Furthermore, in context with the present invention, the expression level of AGTR1 mRNA may be considered as “increased” if the mRNA level of AGTR1 as measured by a method as described herein is at least 1.5-fold or at least 2-fold higher in a biological patient sample (e.g., a breast tissue core biopsy) compared to the AGTR1 mRNA level measured by the same method in a control sample.

Although particularly exemplified by the use of bevacizumab, the invention encompasses the use of other angiogenesis inhibitors such as VEGF-binding agents known in the art for use in combination with standard chemotherapy regimens. The terms “angiogenesis inhibitor” as used herein refers to all agents that alter angiogenesis (e.g., the process of forming blood vessels) and includes agents that block the formation of and/or halt or slow the growth of blood vessels. Non-limiting examples of angiogenesis inhibitors include, in addition to bevacizumab, pegaptanib, sunitinib, sorafenib and vatalanib. Preferably, the angiogenesis inhibitor for use in accordance with the methods of the present invention is bevacizumab. As used herein, the term “bevacizumab” encompass all corresponding anti-VEGF antibodies or anti-VEGF antibody fragments, that fulfil the requirements necessary for obtaining a marketing authorization as an identical or biosimilar product in a country or territory selected from the group of countries or regions consisting of the USA, Europe and Japan.

For use in the detection methods described herein, the skilled person has the ability to label the polypeptides or oligonucleotides encompassed by the present invention. As routinely practiced in the art, hybridization probes for use in detecting mRNA levels and/or antibodies or antibody fragments for use in IHC methods can be labelled and visualized according to standard methods known in the art. Non-limiting examples of commonly used systems include the use of radiolabels, enzyme labels, fluorescent tags, biotin-avidin complexes, chemiluminescence, and the like.

The person skilled in the art, is readily in a position to administer the bevacizumab either alone or in combination with a chemotherapy regimen to the patient/patient group as selected or identified as described herein. In certain contexts, the skilled person may modify, change or amend the administration schemes for the bevacizumab and the chemotherapy regimen in accordance with his/her professional experience.

The present invention relates to a method for the identification of a patient responsive or sensitive to bevacizumab treatment, said patient being suspected to suffer from or being prone to suffer from breast cancer, wherein said method comprises the step of determining the expression level of angiotensin II type 1 receptor (AGTR1) in a biological sample of said patient,

wherein an increased expression level of AGTR1 compared to a control level determined in patients suffering from breast cancer is indicative for said patient to be responsive or sensitive to bevacizumab treatment.

The present invention relates to a method for the identification of a HER2 negative patient responsive or sensitive to bevacizumab treatment, said patient being suspected to suffer from or being prone to suffer from breast cancer, wherein said method comprises the step of determining the expression level of angiotensin II type 1 receptor (AGTR1) in a biological sample of said patient,

wherein an increased expression level of AGTR1 compared to a control level determined in patients suffering from breast cancer is indicative for said patient to be responsive or sensitive to bevacizumab treatment.

The present invention relates to a method for the identification of a HER2 negative patient responsive or sensitive to bevacizumab treatment, said patient being suspected to suffer from or being prone to suffer from locally advanced breast cancer, wherein said method comprises the step of determining the expression level of angiotensin II type 1 receptor (AGTR1) in a biological sample of said patient,

wherein an increased expression level of AGTR1 compared to a control level determined in patients suffering from locally advanced breast cancer is indicative for said patient to be responsive or sensitive to bevacizumab treatment.

The present invention relates to a method for the identification of a patient responsive or sensitive to bevacizumab treatment, said patient being suspected to suffer from or being prone to suffer from a proliferative disorder, wherein said method comprises the step of determining the expression level of angiotensin II type 1 receptor (AGTR1) in a biological sample of said patient,

wherein an increased expression level of AGTR1 compared to a control level determined in patients suffering from the same proliferative disorder is indicative for said patient to be responsive or sensitive to bevacizumab treatment, and
wherein said bevacizumab treatment is comprised in a combination therapy including a chemotherapy.

The present invention relates to a method for the identification of a patient responsive or sensitive to bevacizumab treatment, said patient being suspected to suffer from or being prone to suffer from breast cancer, wherein said method comprises the step of determining the expression level of angiotensin II type 1 receptor (AGTR1) in a biological sample of said patient,

wherein an increased expression level of AGTR1 compared to a control level determined in patients suffering from breast cancer is indicative for said patient to be responsive or sensitive to bevacizumab treatment, and
wherein said bevacizumab treatment is comprised in a combination therapy including a chemotherapy.

The present invention relates to a method for the identification of a HER2 negative patient responsive or sensitive to bevacizumab treatment, said patient being suspected to suffer from or being prone to suffer from breast cancer, wherein said method comprises the step of determining the expression level of angiotensin II type 1 receptor (AGTR1) in a biological sample of said patient,

wherein an increased expression level of AGTR1 compared to a control level determined in patients suffering from breast cancer is indicative for said patient to be responsive or sensitive to bevacizumab treatment, and
wherein said bevacizumab treatment is comprised in a combination therapy including a chemotherapy.

The present invention relates to a method for the identification of a HER2 negative patient responsive or sensitive to bevacizumab treatment, said patient being suspected to suffer from or being prone to suffer from locally advanced breast cancer, wherein said method comprises the step of determining the expression level of angiotensin II type receptor (AGTR1) in a biological sample of said patient,

wherein an increased expression level of AGTR1 compared to a control level determined in patients suffering from locally advanced breast cancer is indicative for said patient to be responsive or sensitive to bevacizumab treatment, and
wherein said bevacizumab treatment is comprised in a combination therapy including a chemotherapy.

The present invention relates to a method for the identification of a patient responsive or sensitive to bevacizumab treatment, said patient being suspected to suffer from or being prone to suffer from a proliferative disorder, wherein said method comprises the step of determining the expression level of angiotensin II type 1 receptor (AGTR1) in a biological sample of said patient,

wherein an increased expression level of AGTR1 compared to a control level determined in patients suffering from the same proliferative disorder is indicative for said patient to be responsive or sensitive to bevacizumab treatment, and
wherein said bevacizumab treatment is comprised in a combination therapy including a therapy with docetaxel.

The present invention relates to a method for the identification of a patient responsive or sensitive to bevacizumab treatment, said patient being suspected to suffer from or being prone to suffer from breast cancer, wherein said method comprises the step of determining the expression level of angiotensin II type 1 receptor (AGTR1) in a biological sample of said patient,

wherein an increased expression level of AGTR1 compared to a control level determined in patients suffering from breast cancer is indicative for said patient to be responsive or sensitive to bevacizumab treatment, and
wherein said bevacizumab treatment is comprised in a combination therapy including a therapy with docetaxel.

The present invention relates to a method for the identification of a HER2 negative patient responsive or sensitive to bevacizumab treatment, said patient being suspected to suffer from or being prone to suffer from breast cancer, wherein said method comprises the step of determining the expression level of angiotensin II type 1 receptor (AGTR1) in a biological sample of said patient,

wherein an increased expression level of AGTR1 compared to a control level determined in patients suffering from breast cancer is indicative for said patient to be responsive or sensitive to bevacizumab treatment, and
wherein said bevacizumab treatment is comprised in a combination therapy including a therapy with docetaxel.

The present invention relates to a method for the identification of a HER2 negative patient responsive or sensitive to bevacizumab treatment, said patient being suspected to suffer from or being prone to suffer from locally advanced breast cancer, wherein said method comprises the step of determining the expression level of angiotensin II type 1 receptor (AGTR1) in a biological sample of said patient,

wherein an increased expression level of AGTR1 compared to a control level determined in patients suffering from locally advanced breast cancer is indicative for said patient to be responsive or sensitive to bevacizumab treatment, and
wherein said bevacizumab treatment is comprised in a combination therapy including a therapy with docetaxel.

The present invention relates to a method for the identification of a patient responsive or sensitive to bevacizumab treatment, said patient being suspected to suffer from or being prone to suffer from a proliferative disorder, wherein said method comprises the step of determining the expression level of angiotensin II type 1 receptor (AGTR1) in a biological sample of said patient,

wherein an increased expression level of AGTR1 compared to a control level determined in patients suffering from the same proliferative disorder is indicative for said patient to be responsive or sensitive to bevacizumab treatment,
wherein said bevacizumab treatment is comprised in a combination therapy including a chemotherapy, and
wherein prior to said bevacizumab treatment, the patient was treated with a chemotherapy without bevacizumab treatment.

The present invention relates to a method for the identification of a patient responsive or sensitive to bevacizumab treatment, said patient being suspected to suffer from or being prone to suffer from breast cancer, wherein said method comprises the step of determining the expression level of angiotensin II type 1 receptor (AGTR1) in a biological sample of said patient,

wherein an increased expression level of AGTR1 compared to a control level determined in patients suffering from breast cancer is indicative for said patient to be responsive or sensitive to bevacizumab treatment,
wherein said bevacizumab treatment is comprised in a combination therapy including a chemotherapy, and
wherein prior to said bevacizumab treatment, the patient was treated with a chemotherapy without bevacizumab treatment.

The present invention relates to a method for the identification of a HER2 negative patient responsive or sensitive to bevacizumab treatment, said patient being suspected to suffer from or being prone to suffer from breast cancer, wherein said method comprises the step of determining the expression level of angiotensin II type 1 receptor (AGTR1) in a biological sample of said patient,

wherein an increased expression level of AGTR1 compared to a control level determined in patients suffering from breast cancer is indicative for said patient to be responsive or sensitive to bevacizumab treatment,
wherein said bevacizumab treatment is comprised in a combination therapy including a chemotherapy, and
wherein prior to said bevacizumab treatment, the patient was treated with a chemotherapy without bevacizumab treatment.

The present invention relates to a method for the identification of a HER2 negative patient responsive or sensitive to bevacizumab treatment, said patient being suspected to suffer from or being prone to suffer from locally advanced breast cancer, wherein said method comprises the step of determining the expression level of angiotensin II type 1 receptor (AGTR1) in a biological sample of said patient,

wherein an increased expression level of AGTR1 compared to a control level determined in patients suffering from locally advanced breast cancer is indicative for said patient to be responsive or sensitive to bevacizumab treatment,
wherein said bevacizumab treatment is comprised in a combination therapy including a chemotherapy, and
wherein prior to said bevacizumab treatment, the patient was treated with a chemotherapy without bevacizumab treatment.

The present invention relates to a method for the identification of a patient responsive or sensitive to bevacizumab treatment, said patient being suspected to suffer from or being prone to suffer from a proliferative disorder, wherein said method comprises the step of determining the expression level of angiotensin II type 1 receptor (AGTR1) in a biological sample of said patient,

wherein an increased expression level of AGTR1 compared to a control level determined in patients suffering from the same proliferative disorder is indicative for said patient to be responsive or sensitive to bevacizumab treatment,
wherein said bevacizumab treatment is comprised in a combination therapy including a chemotherapy, and
wherein prior to said bevacizumab treatment, the patient was treated with a combination of doxorubicin therapy and cyclophosphamide therapy.

The present invention relates to a method for the identification of a patient responsive or sensitive to bevacizumab treatment, said patient being suspected to suffer from or being prone to suffer from breast cancer, wherein said method comprises the step of determining the expression level of angiotensin II type 1 receptor (AGTR1) in a biological sample of said patient,

wherein an increased expression level of AGTR1 compared to a control level determined in patients suffering from breast cancer is indicative for said patient to be responsive or sensitive to bevacizumab treatment,
wherein said bevacizumab treatment is comprised in a combination therapy including a chemotherapy, and
wherein prior to said bevacizumab treatment, the patient was treated with a combination of doxorubicin therapy and cyclophosphamide therapy.

The present invention relates to a method for the identification of a HER2 negative patient responsive or sensitive to bevacizumab treatment, said patient being suspected to suffer from or being prone to suffer from breast cancer, wherein said method comprises the following steps of determining the expression level of angiotensin II type 1 receptor (AGTR1) in a biological sample of said patient,

wherein an increased expression level of AGTR1 compared to a control level determined in patients suffering from breast cancer is indicative for said patient to be responsive or sensitive to bevacizumab treatment,
wherein said bevacizumab treatment is comprised in a combination therapy including a chemotherapy, and
wherein prior to said bevacizumab treatment, the patient was treated with a combination of doxorubicin therapy and cyclophosphamide therapy.

The present invention relates to a method for the identification of a HER2 negative patient responsive or sensitive to bevacizumab treatment, said patient being suspected to suffer from or being prone to suffer from locally advanced breast cancer, wherein said method comprises the step of determining the expression level of angiotensin II type 1 receptor (AGTR1) in a biological sample of said patient,

wherein an increased expression level of AGTR1 compared to a control level determined in patients suffering from locally advanced breast cancer is indicative for said patient to be responsive or sensitive to bevacizumab treatment,
wherein said bevacizumab treatment is comprised in a combination therapy including a chemotherapy, and
wherein prior to said bevacizumab treatment, the patient was treated with a combination of doxorubicin therapy and cyclophosphamide therapy.

The present invention relates to a method for the identification of a patient responsive or sensitive to bevacizumab treatment, said patient being suspected to suffer from or being prone to suffer from a proliferative disorder, wherein said method comprises the step of determining the expression level of angiotensin II type 1 receptor (AGTR1) in a biological sample of said patient,

wherein an increased expression level of AGTR1 compared to a control level determined in patients suffering from the same proliferative disorder is indicative for said patient to be responsive or sensitive to bevacizumab treatment,
wherein said bevacizumab treatment is comprised in a combination therapy including a chemotherapy such as docetaxel therapy, and
wherein prior to said bevacizumab treatment, the patient was treated with a chemotherapy without bevacizumab treatment.

The present invention relates to a method for the identification of a patient responsive or sensitive to bevacizumab treatment, said patient being suspected to suffer from or being prone to suffer from breast cancer, wherein said method comprises the step of determining the expression level of angiotensin II type 1 receptor (AGTR1) in a biological sample of said patient,

wherein an increased expression level of AGTR1 compared to a control level determined in patients suffering from breast cancer is indicative for said patient to be responsive or sensitive to bevacizumab treatment,
wherein said bevacizumab treatment is comprised in a combination therapy including a chemotherapy such as docetaxel therapy, and
wherein prior to said bevacizumab treatment, the patient was treated with a chemotherapy without bevacizumab treatment.

The present invention relates to a method for the identification of a HER2 negative patient responsive or sensitive to bevacizumab treatment, said patient being suspected to suffer from or being prone to suffer from breast cancer, wherein said method comprises the step of determining the expression level of angiotensin II type 1 receptor (AGTR1) in a biological sample of said patient,

wherein an increased expression level of AGTR1 compared to a control level determined in patients suffering from breast cancer is indicative for said patient to be responsive or sensitive to bevacizumab treatment,
wherein said bevacizumab treatment is comprised in a combination therapy including a chemotherapy such as docetaxel therapy, and
wherein prior to said bevacizumab treatment, the patient was treated with a chemotherapy without bevacizumab treatment.

The present invention relates to a method for the identification of a HER2 negative patient responsive or sensitive to bevacizumab treatment, said patient being suspected to suffer from or being prone to suffer from locally advanced breast cancer, wherein said method comprises the step of determining the expression level of angiotensin II type 1 receptor (AGTR1) in a biological sample of said patient,

wherein an increased expression level of AGTR1 compared to a control level determined in patients suffering from locally advanced breast cancer is indicative for said patient to be responsive or sensitive to bevacizumab treatment,
wherein said bevacizumab treatment is comprised in a combination therapy including a chemotherapy such as docetaxel therapy, and
wherein prior to said bevacizumab treatment, the patient was treated with a chemotherapy without bevacizumab treatment.

The present invention relates to a method for the identification of a patient responsive or sensitive to bevacizumab treatment, said patient being suspected to suffer from or being prone to suffer from a proliferative disorder, wherein said method comprises the step of determining the expression level of angiotensin II type 1 receptor (AGTR1) in a biological sample of said patient,

wherein an increased expression level of AGTR1 compared to a control level determined in patients suffering from the same proliferative disorder is indicative for said patient to be responsive or sensitive to bevacizumab treatment,
wherein said bevacizumab treatment is comprised in a combination therapy including a docetaxel therapy, and
wherein prior to said bevacizumab treatment, the patient was treated with a combination of doxorubicin therapy and cyclophosphamide therapy.

The present invention relates to a method for the identification of a patient responsive or sensitive to bevacizumab treatment, said patient being suspected to suffer from or being prone to suffer from breast cancer disorder, wherein said method comprises the step of determining the expression level of angiotensin II type 1 receptor (AGTR1) in a biological sample of said patient,

wherein an increased expression level of AGTR1 compared to a control level determined in patients suffering from breast cancer is indicative for said patient to be responsive or sensitive to bevacizumab treatment,
wherein said bevacizumab treatment is comprised in a combination therapy including a docetaxel therapy, and
wherein prior to said bevacizumab treatment, the patient was treated with a combination of doxorubicin therapy and cyclophosphamide therapy.

The present invention relates to a method for the identification of a HER2 negative patient responsive or sensitive to bevacizumab treatment, said patient being suspected to suffer from or being prone to suffer from breast cancer, wherein said method comprises the step of determining the expression level of angiotensin II type 1 receptor (AGTR1) in a biological sample of said patient,

wherein an increased expression level of AGTR1 compared to a control level determined in patients suffering from breast cancer is indicative for said patient to be responsive or sensitive to bevacizumab treatment,
wherein said bevacizumab treatment is comprised in a combination therapy including a docetaxel therapy, and
wherein prior to said bevacizumab treatment, the patient was treated with a combination of doxorubicin therapy and cyclophosphamide therapy.

The present invention relates to a method for the identification of a HER2 negative patient responsive or sensitive to bevacizumab treatment, said patient being suspected to suffer from or being prone to suffer from locally advanced breast cancer, wherein said method comprises the step of determining the expression level of angiotensin II type 1 receptor (AGTR1) in a biological sample of said patient,

wherein an increased expression level of AGTR1 compared to a control level determined in patients suffering from locally advanced breast cancer is indicative for said patient to be responsive or sensitive to bevacizumab treatment,
wherein said bevacizumab treatment is comprised in a combination therapy including a docetaxel therapy, and
wherein prior to said bevacizumab treatment, the patient was treated with a combination of doxorubicin therapy and cyclophosphamide therapy.

As already mentioned, in accordance with the present invention, the expression level of AGTR1 may be determined before the patient has been treated with bevacizumab and/or any chemotherapy regimen.

### EXAMPLES

The invention is further illustrated by the following non-limiting examples.

### Patients

Seventy-two HER2 negative patients (median age 46 years) diagnosed with locally advanced breast cancer were included in the clinical trial. Treatment consisted of 4 cycles of doxorubicin/cyclophosphamide (60/600 mg/m²) for 21 days, followed by 4 cycles of bevacizumab (15 mg/kg)+docetaxel (75 mg/m²) for 21 days.

### Tumor Evaluation

Physical evaluation of the tumors was performed every 3 weeks. Media tumor size was 4.75 cm. 80.6% were hormone-receptor positive (E: estrogen receptor; P: progesteronreceptor): E+/P+59.7%, E+/P−16.7%, E−/P+4.2%. According to Union Internationale Contre le Cancer (UICC) classification, 20.8% were stadium IIA, 43.1% stadium IIB, 23.6% stadium IIIA, 8.3% stadium IIIB and 4.2% stadium IIIC. Image evaluation was done by mammography and mammary ultrasound and/or magnetic resonance (MR), every 12 weeks.

### Tumor Samples

Tumor samples were collected before treatment for analysis of biomarkers and their relation with pathological complete responders (pCR).

Patients underwent a core-biopsy of the primary tumor for diagnosis and biological characterization of the tumor. At least 3-4 cores had to be collected to allow for routine pathological examinations, immunohistochemical studies and for molecular analysis. At least 1 core of the tumor tissue was snap-frozen in liquid nitrogen and stored at −80° C. until delivery to the central laboratory. Other cores were formalin-fixed and sent to the local laboratory for diagnostic workup

Paraffin embedded specimens were sent (either as block or slices—at least 10) to the central laboratory.

### Exploratory Analysis of Potential Predictive Response Factors

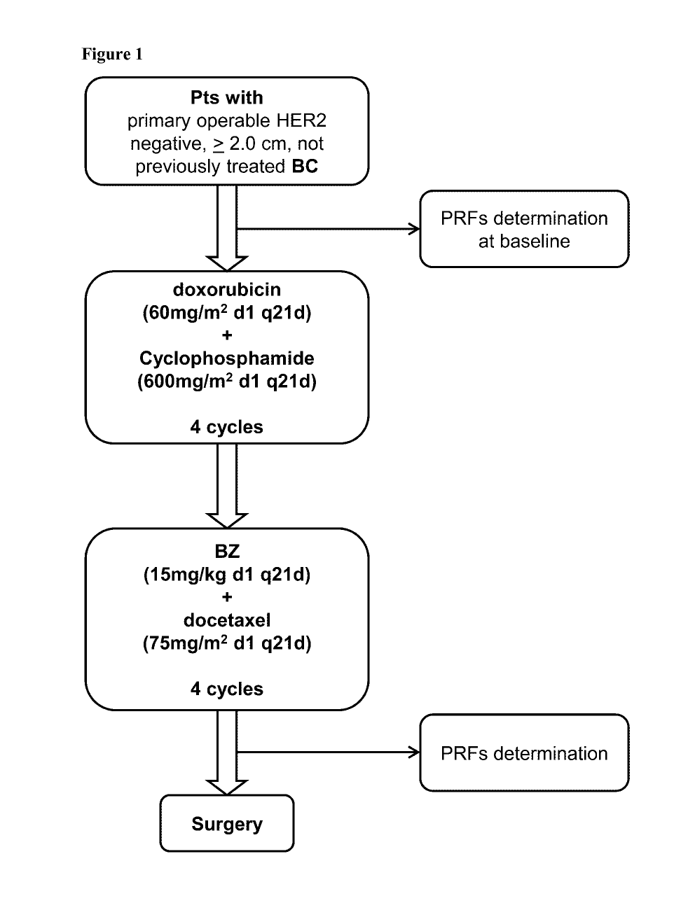

Tables 1-3 shown the results of the analysis made to show the association between several biomarkers in the baseline biopsy and the pathological response to the study treatment. An overview of the procedure is shown in FIG. 1.

The nucleotide and amino acid sequences of the herein used biomarkers disclosed below are: SEQ ID NO: 1 and 2 are the nucleotide and amino acid sequence of human AGTR1, respectively; SEQ ID NO: 3 and 4 are the nucleotide and amino acid sequence of human KISS1, respectively; SEQ ID NO: 5 and 6 are the nucleotide and amino acid sequence of human KISS1R, respectively; SEQ ID NO: 7 and 8 are the nucleotide and amino acid sequence of human VEGF-A, respectively; SEQ ID NO: 9 and 10 are the nucleotide and amino acid sequence of human VEGFR1, respectively; SEQ ID NO: 11 and 12 are the nucleotide and amino acid sequence of human HIF, respectively; and SEQ ID NO: 13 and 14 are the nucleotide and amino acid sequence of human eNOS, respectively.

### Genes/Proteins Evaluated are:

| | |
|---|---|
| human AGTR1 mRNA: GenBank Accession No. AY221090.1 | |
| atgattctcaactcttctactgaagatggtattaaaagaatccaagatgattgtcccaaagctggaaggcataattacatatttgtca | (SEQ ID NO: 1) |
| tgattcctactttatacagtatcatctttgtggtgggaatatttggaaacagcttggtggtgatagtcatttacttttatatgaagct | |
| gaagactgtggccagtgtttttcttttgaatttagcactggctgacttatgctttttactgactttgccactatgggctgtctacaca | |
| gctatggaataccgctggccctttggcaattacctatgtaagattgcttcagccagcgtcagtttcaacctgtacgctagtgtgtttc | |
| tactcacgtgtctcagcattgatcgatacctggctattgttcacccaatgaagtcccgccttcgacgcacaatgcttgtagccaaagt | |
| cacctgcatcatcatttggctgctggcaggcttggccagtttgccagctataatccatcgaaatgtatttttcattgagaacaccaat | |
| attacagtttgtgctttccattatgagtcccaaaattcaacccttccgatagggctgggcctgaccaaaaatatactgggtttcctgt | |
| ttccttttctgatcattcttacaagttatactcttatttggaaggccctaaagaaggcttatgaaattcagaagaacaaaccaagaaa | |
| tgatgatatttttaagataattatggcaattgtgcttttcttttttctttcctggattccccaccaaatattcacttttctggatgta | |
| ttgattcaactaggcatcatacgtgactgtagaattgcagatattgtggacacggccatgcctatcaccatttgtatagcttatttta | |
| acaattgcctgaatcctcttttttatggctttctggggaaaaaatttaaaagatattttctccagcttctaaaatatattcccccaaa | |
| agccaaatcccactcaaacctttcaacaaaaatgagcacgctttcctaccgcccctcagataatgtaagctcatccaccaagaagcct | |
| gcaccatgttttgaggttgagtga | |
| human AGTR1 protein: Swiss Prot Accession No. P30556.1 | |
| MILNSSTEDGIKRIQDDCPKAGRHNYIFVMIPTLYSIIFVVGIFGNSLVVIVIYFYMKLK | (SEQ ID NO: 2) |
| TVASVFLLNLALADLCFLLTLPLWAVYTAMEYRWPFGNYLCKIASASVSFNLYASVF | |
| LLTCLSIDRYLAIVHPMKSRLRRTMLVAKVTCIIIWLLAGLASLPAIIHRNVFFIENTNI | |
| TVCAFHYESQNSTLPIGLGLTKNILGFLFPFLIILTSYTLIWKALKKAYEIQKNKPRND | |
| DIFKIIMAIVLFFFFSWIPHQIFTFLDVLIQLGIIRDCRIADIVDTAMPITICIAYFNNCLNP | |
| LFYGFLGKKFKRYFLQLLKYIPPKAKSHSNLSTKMSTLSYRPSDNVSSSTKKPAPCFEVE | |
| human KISS1 mRNA: GenBank Accession No. AY117143.1 | |
| atgaactcactggtttcttggcagctactgcttttcctctgtgccacccactttggggagccattagaaaaggtggcctctgtggg | (SEQ ID NO: 3) |
| gaattctagacccacaggccagcagctagaatccctgggcctcctggcccccggggagcagagcctgccgtgcaccgagaggaagc | |
| cagctgctactgccaggctgagccgtcgggggacctcgctgtccccgccccccgagagctccgggagccgccagcagccgggcctg | |
| tccgccccccacagccgccagatccccgcaccccagggcgcggtgctggtgcagcgggagaaggacctgccgaactacaactggaa | |
| ctccttcggcctgcgcttcggcaagcgggaggcggcaccagggaaccacggcagaagcgctgggcggggctggggcgcaggtgc | |
| ggggcagtga | |
| human KISS1 protein: Swiss Prot Accession No. Q15726.4 | |
| NSLVSWQLLLFLCATHFGEPLEKVASVGNSRPTGQQLESLGLLAPGEQSLPCTERKP | (SEQ ID NO: 4) |
| AATARLSRRGTSLSPPPESSGSPQQPGLSAPHSRQIPAPQGAVLVQREKDLPNYNWNS | |
| FGLRFGKREAAPGNHGRSAGRG | |
| human KISS1R mRNA: GenBank Accession No. EU883577.1 | |
| atgcacaccgtggctacgtccggacccaacgcgtcctggggggcaccggccaacgcctccggctgcccgggctgtggcgccaac | (SEQ ID NO: 5) |
| gcctcggacggcccagtcccttcgccgcgggccgtggacgcctggctcgtgccgctcttcttcgcggcgctgatgctgctgggcctg | |
| gtggggaactcgctggtcatctacgtcatctgccgccacaagccgatgcggaccgtgaccaacttctacatcgccaacctggcggcc | |
| acggacgtgaccttcctcctgtgctgcgtccccttcacggccctgctgtacccgctgcccggctgggtgctgggcgacttcatgtgc | |
| aagttcgtcaactacatccagcaggtctcggtgcaggccacgtgtgccactctgaccgccatgagtgtggaccgctggtacgtgacg | |
| gtgttcccgttgcgcgccctgcaccgccgcacgccccgcctggcgctggctgtcagcctcagcatctgggtaggctctgcggcggtg | |
| tctgcgccggtgctcgccctgcaccgcctgtcacccgggccgcgcgcctactgcagtgaggccttccccagccgcgccctggagcgc | |
| gccttcgcactgtacaacctgctggcgctgtacctgctgccgctgctcgccacctgcgcctgctatgcggccatgctgcgccacctg | |
| ggccgggtcgccgtgcgccccgcgcccgccgatagcgccctgcaggggcaggtgctggcagagcgcgcaggcgccgtgcgggcc | |
| aaggtctcgcggctggtggcggccgtggtcctgctcttcgccgcctgctggggccccatccagctgttcctggtgctgcaggcgctg | |
| ggccccgcgggctcctggcacccacgcagctacgccgcctacgcgcttaagacctgggctcactgcatgtcctacagcaactccgcg | |
| ctgaacccgctgctctacgccttcctgggctcgcacttccgacaggccttccgccgcgtctgcccctgcgcgccgcgccgcccccgc | |
| cgcccccgccggcccggaccctcggaccccgcagccccacacgcggagctgctccgcctggggtcccacccggcccccgccag | |
| ggcgcagaagccagggagcagtgggctggccgcgcgcgggctgtgcgtcctgggggaggacaacgcccctctctga | |
| human KISS1R protein: Swiss Prot Accession No. Q969F8.2 | |
| MHTVATSGPNASWGAPANASGCPGCGANASDGPVPSPRAVDAWLVPLFFAALMLL | (SEQ ID NO: 6) |
| GLVGNSLVIYVICRHKPMRTVTNFYIANLAATDVTFLLCCVPFTALLYPLPGWVLGD | |
| FMCKFVNYIQQVSVQATCATLTAMSVDRWYVTVFPLRALHRRTPRLALAVSLSIWV | |
| GSAAVSAPVLALHRLSPGPRAYCSEAFPSRALERAFALYNLLALYLLPLLATCACYA | |
| AMLRHLGRVAVRPAPADSALQGQVLAERAGAVRAKVSRLVAAVVLLFAACWGPIQ | |
| LFLVLQALGPAGSWHPRSYAAYALKTWAHCMSYSNSALNPLLYAFLGSHFRQAFRR | |
| VCPCAPRRPRRPRRPGPSDPAAPHAELLRLGSHPAPARAQKPGSSGLAARGLCVLGE | |
| DNAPL | |
| human VEGF-A mRNA: GenBank Accession No. M32977.1 | |
| agtgtgctggcggcccggcgcgagccggcccggccccggtcgggcctccgaaaccatgaactttctgctgtcttgggtgcattggag | (SEQ ID NO: 7) |
| cctcgccttgctgctctacctccaccatgccaagtggtcccaggctgcacccatggcagaaggaggagggcagaatcatcacgaagt | |
| ggtgaagttcatggatgtctatcagcgcagctactgccatccaatcgagaccctggtggacatcttccaggagtaccctgatgagat | |
| cgagtacatcttcaagccatcctgtgtgcccctgatgcgatgcgggggctgctgcaatgacgagggcctggagtgtgtgcccactga | |
| ggagtccaacatcaccatgcagattatgcggatcaaacctcaccaaggccagcacataggagagatgagcttcctacagcacaacaa | |
| atgtgaatgcagaccaaagaaagatagagcaagacaagaaaatccctgtgggccttgctcagagcggagaaagcatttgtttgtaca | |
| agatccgcagacgtgtaaatgttcctgcaaaaacacagactcgcgttgcaaggcgaggcagcttgagttaaacgaacgtacttgcag | |
| atgtgacaagccgaggcggtgagccgggcaggaggaaggagcctccctcagggtttcgggaaccagatctctcaccaggaaagactg | |
| atacagaacgatcgatacagaaaccacgctgccgccaccacaccatcaccatcgacagaacagtccttaatccagaaacctgaaatg | |
| aaggaagaggagactctgcgcagagcactttgggtccggagggcgagactccggcggaagcattcccgggcgggtgacccagcacgg | |
| tccctcttggaattggattcgccattttatttttcttgctgctaaatcaccgagcccggaagattagagagttttatttctgggatt | |
| cctgtagacacaccgcggccgccagcacactg | |
| human VEGF-A protein: Swiss Prot Accession No. P15692.2 | |
| MNFLLSWVHWSLALLLYLHHAKWSQAAPMAEGGGQNHHEVVKFMDVYQRSYCH | (SEQ ID NO: 8) |
| PIETLVDIFQEYPDEIEYIFKPSCVPLMRCGGCCNDEGLECVPTEESNITMQIMRIKPHQ | |
| GQHIGEMSFLQHNKCECRPKKDRARQEKKSVRGKGKGQKRKRKKSRYKSWSVYV | |
| GARCCLMPWSLPGPHPCGPCSERRKHLFVQDPQTCKCSCKNTDSRCKARQLELNER | |
| TCRCDKPRR | |
| human VEGFR1 mRNA: GenBank Accession No. AF063657.2 | |
| atggtcagctactgggacaccggggtcctgctgtgcgcgctgctcagctgtctgcttctcacaggatctagttcaggttcaaaatta | (SEQ ID NO: 9) |
| aaagatcctgaactgagtttaaaaggcacccagcacatcatgcaagcaggccagacactgcatctccaatgcaggggggaagcagcc | |
| cataaatggtctttgcctgaaatggtgagtaaggaaagcgaaaggctgagcataactaaatctgcctgtggaagaaatggcaaacaa | |
| ttctgcagtactttaaccttgaacacagctcaagcaaaccacactggcttctacagctgcaaatatctagctgtacctacttcaaag | |
| aagaaggaaacagaatctgcaatctatatatttattagtgatacaggtagacctttcgtagagatgtacagtgaaatccccgaaatt | |
| atacacatgactgaaggaagggagctcgtcattccctgccgggttacgtcacctaacatcactgttactttaaaaaagtttccactt | |
| gacactttgatccctgatggaaaacgcataatctgggacagtagaaagggcttcatcatatcaaatgcaacgtacaaagaaataggg | |
| cttctgacctgtgaagcaacagtcaatgggcatttgtataagacaaactatctcacacatcgacaaaccaatacaatcatagatgtc | |
| caaataagcacaccacgcccagtcaaattacttagaggccatactcttgtcctcaattgtactgctaccactcccttgaacacgaga | |
| gttcaaatgacctggagttaccctgatgaaaaaaataagagagcttccgtaaggcgacgaattgaccaaagcaattcccatgccaac | |
| atattctacagtgttcttactattgacaaaatgcagaacaaagacaaaggactttatacttgtcgtgtaaggagtggaccatcattc | |
| aaatctgttaacacctcagtgcatatatatgataaagcattcatcactgtgaaacatcgaaaacagcaggtgcttgaaaccgtagct | |
| ggcaagcggtcttaccggctctctatgaaagtgaaggcatttccctcgccggaagttgtatggttaaaagatgggttacctgcgact | |
| gagaaatctgctcgctatttgactcgatggctactcgttattatcaaggacgtaactgaagaggatgcagggaattatacaatcttg | |
| ctgagcataaaacagtcaaatgtgtttaaaaacctcactgccactctaattgtcaatgtgaaaccccagatttacgaaaaggccgtg | |
| tcatcgtttccagacccggctctctacccactgggcagcagacaaatcctgacttgtaccgcatatggtatccctcaacctacaatc | |
| aagtggttctggcacccctgtaaccataatcattccgaagcaaggtgtgacttttgttccaataatgaagagtcctctatcctggat | |
| gctgacagcaacatgggaaacagaattgagagcatcactcagcgcatggcaataatagaaggaaagaataagatggctagcaccttg | |
| gttgtggctgactctagaatttctggaatctacatttgcatagcttccaataaagttgggactgtgggaagaaacataagcttttat | |
| atcacagatgtgccaaatgggtttcatgttaacttggaaaaaatgccgacggaaggagaggacctgaaactgtcttgcacagttaac | |
| aagttcttatacagagacgttacttggattttactgcggacagttaataacagaacaatgcactacagtattagcaagcaaaaaatg | |
| gccatcactaaggagcactccatcactcttaatcttaccatcatgaatgtttccctgcaagattcaggcacctatgcctgcagagcc | |
| aggaatgtatacacaggggaagaaatcctccagaagaaagaaattacaatcagagatcaggaagcaccatacctcctgcgaaacctc | |
| agtgatcacacagtggccatcagcagttccaccactttagactgtcatgctaatggtgtccccgagcctcagatcacttggtttaaa | |
| aacaaccacaaaatacaacaagagcctggaattattttaggaccaggaagcagcacgctgtttattgaaagagtcacagaagaggat | |
| gaaggtgtctatcactgcaaagccaccaaccagaagggctctgtggaaagttcagcatacctcactgttcaaggaacctcggacaag | |
| tctaatctggagctgatcactctaacatgcacctgtgtggctgcgactctcttctggctcctattaaccctctttatccgaaaaatg | |
| aaaaggtcttcttctgaaataaagactgactacctatcaattataatggacccagatgaagttcctttggatgagcagtgtgagcgg | |
| ctcccttatgatgccagcaagtgggagtttgcccgggagagacttaaactgggcaaatcacttggaagaggggcttttggaaaagtg | |
| gttcaagcatcagcatttggcattaagaaatcacctacgtgccggactgtggctgtgaaaatgctgaaagaggggggccacggccac | |
| gagtacaaagctctgatgactgagctaaaaatcttgacccacattggccaccatctgaacgtggttaacctgctgggagcctgcacc | |
| aagcaaggagggcctctgatggtgattgttgaatactgcaaatatggaaatctctccaactacctcaagagcaaacgtgacttattt | |
| tttctcaacaaggatgcagcactacacatggagcctaagaaagaaaaaatggagccaggcctggaacaaggcaagaaaccaagaact | |
| agatgcgtcaccagcagcgaaagctttgcgagctccggctttcaggaagataaaagtctgagtgatgttgaggaagaggaggattct | |
| gacggtttctacaaggagcccatcactatggaagatctgatttcttacagttttcaagtggccagaggcatggagttcctgtcttcc | |
| agaaagtgcattcatcgggacctggcagcgagaaacattcttttatctgagaacaacgtggtgaagatttgtgattttggccttgcc | |
| cgggatatttataagaaccccgattatgtgagaaaaggagatactcgacttcctctgaaatggatggctcctgaatctatctttgac | |
| aaaatctacagcaccaagagcgacgtgtggtcttacggagtattgctgtgggaaatcttctccttaggtgggtctccatacccagga | |
| gtacaaatggatgaggacttttgcagtcgcctgagggaaggcatgaggatgagagctcctgagtactctactcctgaaatctatcag | |
| atcatgctggactgctggcacagagacccaaaagaaaggccaagatttgcagaacttgtggaaaaactaggtgatttgcttcaagca | |
| aatgtacaacaggatggtaaagactacatcccaatcaatgccatactgacaggaaatagtgggtttacatactcaactcctgccttc | |
| tctgaggacttcttcaaggaaagtatttcagctccgaagtttaattcaggaagctctgatgatgtcagatatgtaaatgctttcaag | |
| ttcatgagcctggaaagaatcaaaacctttgaagaacttttaccgaatgccacctccatgtttgatgactaccagggcgacagcagc | |
| actctgttggcctctcccatgctgaagcgcttcacctggactgacagcaaacccaaggcctcgctcaagattgacttgagagtaacc | |
| agtaaaagtaaggagtcggggctgtctgatgtcagcaggcccagtttctgccattccagctgtgggcacgtcagcgaaggcaagcgc | |
| aggttcacctacgaccacgctgagctggaaaggaaaatcgcgtgctgctccccgcccccagactacaactcggtggtcctgtactcc | |
| accccacccatctag | |
| human VEGFR1 protein: GenBank Accession No: AF063657.2 | |
| MVSYWDTGVLLCALLSCLLLTGSSSGSKLKDPELSLKGTQHIMQAGQTLHLQCRGE | (SEQ ID NO: 10) |
| AAHKWSLPEMVSKESERLSITKSACGRNGKQFCSTLTLNTAQANHTGFYSCKYLAV | |
| PTSKKKETESAIYIFISDTGRPFVEMYSEIPEIIHMTEGRELVIPCRVTSPNITVTLKKFPL | |
| DTLIPDGKRIIWDSRKGFIISNATYKEIGLLTCEATVNGHLYKTNYLTHRQTNTIIDVQI | |
| STPRPVKLLRGHTLVLNCTATTPLNTRVQMTWSYPDEKNKRASVRRRIDQSNSHANI | |
| FYSVLTIDKMQNKDKGLYTCRVRSGPSFKSVNTSVHIYDKAFITVKHRKQQVLETVA | |
| GKRSYRLSMKVKAFPSPEVVWLKDGLPATEKSARYLTRGYSLIIKDVTEEDAGNYTI | |
| LLSIKQSNVFKNLTATLIVNVKPQIYEKAVSSFPDPALYPLGSRQILTCTAYGIPQPTIK | |
| WFWHPCNHNHSEARCDFCSNNEESSILDADSNMGNRIESITQRMAIIEGKNKMASTL | |
| VVADSRISGIYICIASNKVGTVGRNISFYITDVPNGFHVNLEKMPTEGEDLKLSCTVN | |
| KFLYRDVTWILLRTVNNRTMHYSISKQKMAITKEHSITLNLTIMNVSLQDSGTYACR | |
| ARNVYTGEEILQKKEITIRDQEAPYLLRNLSDHTVAISSSTTLDCHANGVPEPQITWFK | |
| NNHKIQQEPGIILGPGSSTLFIERVTEEDEGVYHCKATNQKGSVESSAYLTVQGTSDK | |
| SNLELITLTCTCVAATLFWLLLTLFIRKMKRSSSEIKTDYLSIIMDPDEVPLDEQCERLP | |
| YDASKWEFARERLKLGKSLGRGAFGKVVQASAFGIKKSPTCRTVAVKMLKEGATAS | |
| EYKALMTELKILTHIGHHLNVVNLLGACTKQGGPLMVIVEYCKYGNLSNYLKSKRD | |
| LFFLNKDAALHMEPKKEKMEPGLEQGKKPRLDSVTSSESFASSGFQEDKSLSDVEEE | |
| EDSDGFYKEPITMEDLISYSFQVARGMEFLSSRKCIHRDLAARNILLSENNVVKICDF | |
| GLARDIYKNPDYVRKGDTRLPLKWMAPESIFDKIYSTKSDVWSYGVLLWEIFSLGGS | |
| PYPGVQMDEDFCSRLREGMRMRAPEYSTPEIYQIMLDCWHRDPKERPRFAELVEKL | |
| GDLLQANVQQDGKDYIPINAILTGNSGFTYSTPAFSEDFFKESISAPKFNSGSSDDVRY | |
| VNAFKFMSLERIKTFEELLPNATSMFDDYQGDSSTLLASPMLKRFTWTDSKPKASLKI | |
| DLRVTSKSKESGLSDVSRPSFCHSSCGHVSEGKRRFTYDHAELERKIACCSPPPDYNS | |
| VVLYSTPPI | |
| human HIF mRNA: GenBank Accession No. BT009776.1 | |
| atggagggcgccggcggcgcgaacgacaagaaaaagataagttctgaacgtcgaaaagaaaagtctcgagatgcagccagatctcgg | (SEQ ID NO: 11) |
| cgaagtaaagaatctgaagttttttatgagcttgctcatcagttgccacttccacataatgtgagttcgcatcttgataaggcctct | |
| gtgatgaggcttaccatcagctatttgcgtgtgaggaaacttctggatgctggtgatttggatattgaagatgacatgaaagcacag | |
| atgaattgcttttatttgaaagccttggatggttttgttatggttctcacagatgatggtgacatgatttacatttctgataatgtg | |
| aacaaatacatgggattaactcagtttgaactaactggacacagtgtgtttgattttactcatccatgtgaccatgaggaaatgaga | |
| gaaatgcttacacacagaaatggccttgtgaaaaagggtaaagaacaaaacacacagcgaagcttttttctcagaatgaagtgtacc | |
| ctaactagccgaggaagaactatgaacataaagtctgcaacatggaaggtattgcactgcacaggccacattcacgtatatgatacc | |
| aacagtaaccaacctcagtgtgggtataagaaaccacctatgacctgcttggtgctgatttgtgaacccattcctcacccatcaaat | |
| attgaaattcctttagatagcaagactttcctcagtcgacacagcctggatatgaaattttcttattgtgatgaaagaattaccgaa | |
| ttgatgggatatgagccagaagaacttttaggccgctcaatttatgaatattatcatgctttggactctgatcatctgaccaaaact | |
| catcatgatatgtttactaaaggacaagtcaccacaggacagtacaggatgcttgccaaaagaggtggatatgtctgggttgaaact | |
| caagcaactgtcatatataacaccaagaattctcaaccacagtgcattgtatgtgtgaattacgttgtgagtggtattattcagcac | |
| gacttgattttctcccttcaacaaacagaatgtgtccttaaaccggttgaatcttcagatatgaaaatgactcagctattcaccaaa | |
| gttgaatcagaagatacaagtagcctctttgacaaacttaagaaggaacctgatgctttaactttgctggccccagccgctggagac | |
| acaatcatatctttagattttggcagcaacgacacagaaactgatgaccagcaacttgaggaagtaccattatataatgatgtaatg | |
| ctcccctcacccaacgaaaaattacagaatataaatttggcaatgtctccattacccaccgctgaaacgccaaagccacttcgaagt | |
| agtgctgaccctgcactcaatcaagaagttgcattaaaattagaaccaaatccagagtcactggaactttcttttaccatgccccag | |
| attcaggatcagacacctagtccttccgatggaagcactagacaaagttcacctgagcctaatagtcccagtgaatattgtttttat | |
| gtggatagtgatatggtcaatgaattcaagttggaattggtagaaaaactttttgctgaagacacagaagcaaagaacccattttct | |
| actcaggacacagatttagacttggagatgttagctccctatatcccaatggatgatgacttccagttacgttccttcgatcagttg | |
| tcaccattagaaagcagttccgcaagccctgaaagcgcaagtcctcaaagcacagttacagtattccagcagactcaaatacaagaa | |
| cctactgctaatgccaccactaccactgccaccactgatgaattaaaaacagtgacaaaagaccgtatggaagacattaaaatattg | |
| attgcatctccatctcctacccacatacataaagaaactactagtgccacatcatcaccatatagagatactcaaagtcggacagcc | |
| tcaccaaacagagcaggaaaaggagtcatagaacagacagaaaaatctcatccaagaagccctaacgtgttatctgtcgctttgagt | |
| caaagaactacagttcctgaggaagaactaaatccaaagatactagctttgcagaatgctcagagaaagcgaaaaatggaacatgat | |
| ggttcactttttcaagcagtaggaattggaacattattacagcagccagacgatcatgcagctactacatcactttcttggaaacgt | |
| gtaaaaggatgcaaatctagtgaacagaatggaatggagcaaaagacaattattttaataccctctgatttagcatgtagactgctg | |
| gggcaatcaatggatgaaagtggattaccacagctgaccagttatgattgtgaagttaatgctcctatacaaggcagcagaaaccta | |
| ctgcagggtgaagaattactcagagctttggatcaagttaactag | |
| human HIF protein: Swiss Prot Accession No. Q16665.1 | |
| EGAGGANDKKKISSERRKEKSRDAARSRRSKESEVFYELAHQLPLPHNVSSHLDKAS | (SEQ ID NO: 12) |
| VMRLTISYLRVRKLLDAGDLDIEDDMKAQMNCFYLKALDGFVMVLTDDGDMIYIS | |
| DNVNKYMGLTQFELTGHSVFDFTHPCDHEEMREMLTHRNGLVKKGKEQNTQRSFF | |
| LRMKCTLTSRGRTMNIKSATWKVLHCTGHIHVYDTNSNQPQCGYKKPPMTCLVLIC | |
| EPIPHPSNIEIPLDSKTFLSRHSLDMKFSYCDERITELMGYEPEELLGRSIYEYYHALDS | |
| DHLTKTHHDMFTKGQVTTGQYRMLAKRGGYVWVETQATVIYNTKNSQPQCIVCVN | |
| YVVSGIIQHDLIFSLQQTECVLKPVESSDMKMTQLFTKVESEDTSSLFDKLKKEPDAL | |
| TLLAPAAGDTIISLDFGSNDTETDDQQLEEVPLYNDVMLPSPNEKLQNINLAMSPLPT | |
| AETPKPLRSSADPALNQEVALKLEPNPESLELSFTMPQIQDQTPSPSDGSTRQSSPEPN | |
| SPSEYCFYVDSDMVNEFKLELVEKLFAEDTEAKNPFSTQDTDLDLEMLAPYIPMDDD | |
| FQLRSFDQLSPLESSSASPESASPQSTVTVFQQTQIQEPTANATTTTATTDELKTVTKD | |
| RMEDIKILIASPSPTHIHKETTSATSSPYRDTQSRTASPNRAGKGVIEQTEKSHPRSPNV | |
| LSVALSQRTTVPEEELNPKILALQNAQRKRKMEHDGSLFQAVGIGTLLQQPDDHAAT | |
| TSLSWKRVKGCKSSEQNGMEQKTIILIPSDLACRLLGQSMDESGLPQLTSYDCEVNA | |
| PIQGSRNLLQGEELLRALDQVN | |
| human eNOS mRNA: GenBank Accession No. AF400594.1 | |
| atgggcaacttgaagagcgtggcccaggagcctgggccaccctgcggcctggggctggggctgggccttgggctgtgcggcaag | (SEQ ID NO: 13) |
| cagggcccagccaccccggcccctgagcccagccgggccccagcatccctactcccaccagcgccagaacacagccccccgag | |
| ctccccgctaacccagcccccagaggggcccaagttccctcgtgtgaagaactgggaggtggggagcatcacctatgacaccctca | |
| gcgcccaggcgcagcaggatgggccctgcaccccaagacgctgcctgggctccctggtatttccacggaaactacagggccggcc | |
| ctcccccggccccccggcccctgagcagctgctgagtcaggcccgggacttcatcaaccagtactacagctccattaagaggagcg | |
| gctcccaggcccacgaacagcggcttcaagaggtggaagccgaggtggcagccacaggcacctaccagcttagggagagcgagc | |
| tggtgttcggggctaagcaggcctggcgcaacgctccccgctgcgtgggccggatccagtgggggaagctgcaggtgttcgatgcc | |
| cgggactgcaggtctgcacaggaaatgttcacctacatctgcaaccacatcaagtatgccaccaaccggggcaaccttcgctcggcc | |
| atcacagtgttcccgcagcgctgccctggccgaggagacttccgaatctggaacagccagctggtgcgctacgcgggctaccggca | |
| gcaggacggctctgtgcggggggacccagccaacgtggagatcaccgagctctgcattcagcacggctggaccccaggaaacggt | |
| cgcttcgacgtgctgcccctgctgctgcaggccccagatgagcccccagaactcttccttctgccccccgagctggtccttgaggtgc | |
| ccctggagcaccccacgctggagtggtttgcagccctgggcctgcgctggtacgccctcccggcagtgtccaacatgctgctggaaa | |
| ttgggggcctggagttccccgcagcccccttcagtggctggtacatgagcactgagatcggcacgaggaacctgtgtgaccctcacc | |
| gctacaacatcctggaggatgtggctgtctgcatggacctggatacccggaccacctcgtccctgtggaaagacaaggcagcagtgg | |
| aaatcaacgtggccgtgctgcacagttaccagctagccaaagtcaccatcgtggaccaccacgccgccacggcctctttcatgaagc | |
| acctggagaatgcgcagaaggccagggggggctgccctgcagactgggcctggatcgtgccccccatctcgggcagcctcactcc | |
| tgttttccatcaggagatggtcaactatttcctgtccccggccttccgctaccagccagacccctggaaggggagtgccgccaagggc | |
| accggcatcaccaggaagaagacctttaaagaagtggccaacgccgtgaagatctccgcctcgctcatgggcacggtgatggcgaa | |
| gcgagtgaaggcgacaatcctgtatggctccgagaccggccgggcccagagctacgcacagcagctggggagactcttccggaag | |
| gcttttgatccccgggtcctgtgtatggatgagtatgacgtggtgtccctcgaacacgagacgctggtgctggtggtaaccagcacat | |
| ttgggaatggggatcccccggagaatggagagagctttgcagctgccctgatggagatgtccggcccctacaacagctcccctcggcc | |
| ggaacagcacaagagttataagatccgcttcaacagcatctcctgctcagacccactggtgtcctcttggcggcggaagaggaagga | |
| gtccagtaacacagacagtgcaggggccctgggcaccctcaggttctgtgtgttcgggctcggctcccgggcatacccccacttctgc | |
| gcctttgctcgtgccgtggacacacggctggaggaactgggcggggagcggctgctgcagctgggccagggcgacgagctgtgc | |
| ggccaggaggaggccttccgaggctgggcccaggctgccttccaggccgcctgtgagaccttctgtgtgggagaggatgccaagg | |
| ccgccgcccgagacatcttcagccccaaacggagctggaagcgccagaggtaccggctgagcgcccaggccgagggcctgcagt | |
| tgctgccaggtctgatccacgtgcacaggcggaagatgttccaggctacaatccgctcagtggaaaacctgcaaagcagcaagtcca | |
| cgagggccaccatcctggtgcgcctggacaccggaggccaggaggggctgcagtaccagccgggggaccacataggtgtctgcc | |
| cgcccaaccggcccggccttgtggaggcgctgctgagccgcgtggaggacccgccggcgcccactgagcccgtggcagtagagc | |
| agctggagaagggcagccctggtggccctccccccggctgggtgcgggacccccggctgcccccgtgcacgctgcgccaggctc | |
| tcaccttcttcctggacatcacctccccacccagccctcagctcttgcggctgctcagcaccttggcagaagagcccagggaacagca | |
| ggagctggaggccctcagccaggatccccgacgctacgaggagtggaagtggttccgctgccccacgctgctggaggtgctggag | |
| cagttcccgtcggtggcgctgcctgccccactgctcctcacccagctgcctctgctccagccccggtactactcagtcagctcggcac | |
| ccagcacccacccaggagagatccacctcactgtagctgtgctggcatacaggactcaggatgggctgggccccctgcactatgga | |
| gtctgctccacgtggctaagccagctcaagcccggagaccctgtgccctgcttcatccggggggctccctccttccggctgccacccg | |
| atcccagcttgccctgcatcctggtgggtccaggcactggcattgcccccttccggggattctggcaggagcggctgcatgacattga | |
| gagcaaagggctgcagcccactcccatgactttggtgttcggctgccgatgctcccaacttgaccatctctaccgcgacgaggtgcag | |
| aacgcccagcagcgcggggtgtttggccgagtcctcaccgccttctcccgggaacctgacaaccccaagacctacgtgcaggacat | |
| cctgaggacggagctggctgcggaggtgcaccgcgtgctgtgcctcgagcggggccacatgtttgtctgcggcgatgttaccatggc | |
| aaccaacgtcctgcagaccgtgcagcgcatcctggcgacggagggcgacatggagctggacgaggccggcgacgtcatggcgt | |
| gctgcgggatcagcaacgctaccacgaagacattttcgggctcacgctgcgcacccaggaggtgacaagccgcatacgcacccag | |
| agcttttccttgcaggagcgtcagttgcggggcgcagtgccctgggcgttcgaccctcccggctcagacaccaacagcccctga | |
| human eNOS protein: Swiss Prot Accession NO. P29474.3 | |
| MGNLKSVAQEPGPPCGLGLGLGLGLCGKQGPATPAPEPSRAPASLLPPAPEHSPPSSP | (SEQ ID NO: 14) |
| LTQPPEGPKFPRVKNWEVGSITYDTLSAQAQQDGPCTPRRCLGSLVFPRKLQGRPSP | |
| GPPAPEQLLSQARDFINQYYSSIKRSGSQAHEQRLQEVEAEVAATGTYQLRESELVFG | |
| AKQAWRNAPRCVGRIQWGKLQVFDARDCRSAQEMFTYICNHIKYATNRGNLRSAIT | |
| VFPQRCPGRGDFRIWNSQLVRYAGYRQQDGSVRGDPANVEITELCIQHGWTPGNGR | |
| FDVLPLLLQAPDEPPELFLLPPELVLEVPLEHPTLEWFAALGLRWYALPAVSNMLLEI | |
| GGLEFPAAPFSGWYMSTEIGTRNLCDPHRYNILEDVAVCMDLDTRTTSSLWKDKAA | |
| VEINVAVLHSYQLAKVTIVDHHAATASFMKHLENEQKARGGCPADWAWIVPPISGS | |
| LTPVFHQEMVNYFLSPAFRYQPDPWKGSAAKGTGITRKKTFKEVANAVKISASLMG | |
| TVMAKRVKATILYGSETGRAQSYAQQLGRLFRKAFDPRVLCMDEYDVVSLEHETLV | |
| LVVTSTFGNGDPPENGESFAAALMEMSGPYNSSPRPEQHKSYKIRFNSISCSDPLVSS | |
| WRRKRKESSNTDSAGALGTLRFCVFGLGSRAYPHFCAFARAVDTRLEELGGERLLQ | |
| LGQGDELCGQEEAFRGWAQAAFQAACETFCVGEDAKAAARDIFSPKRSWKRQRYR | |
| LSAQAEGLQLLPGLIHVHRRKMFQATIRSVENLQSSKSTRATILVRLDTGGQEGLQY | |
| QPGDHIGVCPPNRPGLVEALLSRVEDPPAPTEPVAVEQLEKGSPGGPPPGWVRDPRLP | |
| PCTLRQALTFFLDITSPPSPQLLRLLSTLAEEPREQQELEALSQDPRRYEEWKWFRCPT | |
| LLEVLEQFPSVALPAPLLLTQLPLLQPRYYSVSSAPSTHPGEIHLTVAVLAYRTQDGL | |
| GPLHYGVCSTWLSQLKPGDPVPCFIRGAPSFRLPPDPSLPCILVGPGTGIAPFRGFWQE | |
| RLHDIESKGLQPTPMTLVFGCRCSQLDHLYRDEVQNAQQRGVFGRVLTAFSREPDNP | |
| KTYVQDILRTELAAEVHRVLCLERGHMFVCGDVTMATNVLQTVQRILATEGDMEL | |
| DEAGDVIGVLRDQQRYHEDIFGLTLRTQEVTSRIRTQSFSLQERQLRGAVPWAFDPP | |
| GSDTNSP | |

Biomarker amplification as shown in Table 1 was measured by FISH (fluorescence in situ hybridization). Protein expression was measured by IHC (immunohistochemistry) analysis.

### Immunohistochemistry (IHC) was Performed as Follows.

Tissue sections were fixed in formalin and embedded in paraffin blocks according to standard procedures. Glass slides were cleaned with 95% ethanol, treated with subbing solution and air dried. Alternatively, pre-treated slides can be used. Subsequently, 4 to 6 micron thick tissue sections were cut, applied to the slides and deparaffinised in xylenes using three changes for 5 min each. Sections were gradually hydrated through graded alcohols: washing twice for 10 min in 100% ethanol, followed by washin twice for 10 min in 95% ethanol, followed by washing in deionized H₂O for 1 min under stirring. Excess liquid was aspirated from the slides.

Antigen unmasking (heat treatment): Slides were placed in a container into a microwave oven and covered with EDTA 1 mM (pH 8.0).

Immunofluorescence staining: After each step, reagents were removed by suction. Sufficient reagent was used to ensure coverage of the specimen (app. 100-500 μl per slide). Subsequently, specimens were incubated with 10% normal blocking serum in PBS (derived from the same species as the secondary antibody) for 20 min to suppress non-specific binding of IgG followed by incubation with primary antibody (Santa Cruz Biotechnology, USA, antibody sc-1173) for 60 min, followed by washing three times with PBS for 5 min, incubation with AlexaFluor-488 (Molecular Probes, Invitrogen) for 45 min in a dark chamber and extensive washing with PBS. Then, the coverslip was mounted with DAPI for 30 min at 4° C.

Gene Expression (mRNA) was Measured by RT-PCR Analysis as Follows.

Sample total RNA extraction: RNA was extracted using the extraction protocol of Qiagen “RNeasy FFPE Kit”. The extraction process was done by automatic robot Qiacube. All samples were cut in 10 sections of 5 micra.

Quantification of extracted RNA was done using a Nanodrop® spectrophotometer. Samples exhibiting values of a ratio A260/A280 equal or higher than 1.8 and those exhibiting values of a ratio A260/230 equal or higher than 1.7 were considered appropriate for the expressions study.

For the selection of endogen genes or housekeeping genes, an expression array from SABiosciences, USA, was performed that permits the amplification of one single plate for a total of 8 samples of 12 endogen genes (Housekeeping Genes PCR Array Human Housekeeping Genes RT2, ref 103PAHS-000A-2, SABiosciences). The samples used for the search of the housekeeping were pre-amplified using the primers “Primer Mix for Human Housekeeping Genes RT2 FFPE PreAMP” obtained from SABiosciences.

Genomic DNA cleaning, reverse transcription of the extracted RNA, pre-amplification and RT-PCR for the proposed genes in the study were done using the kit “RT2 FFPE PreAMP cDNA Synthesis Kit FFPE RNA samples” (C-07, obtained from SABiosciences), personalized expression plates 103CAPH09806A-12 (obtained from SABiosciences) and peronalized primers for the pre-amplification “RT2 Custom Nano PreAMP Primer Mix, ref CAPH09806-12” (obtained from SABiosciences). The process was done starting from 500 ng of RNA for all samples.

PCR process at real time was done by ABIPrism 7000 Sequence Detector System.

TABLE 1
Biomarkers Amplification

| Biomarker | Analyzed cases | pCR (N, %) | p-value |
|---|---|---|---|
| KISS1 | 23 | | 0.4864 |
| aneuploid | 8 | 1 (12.5%) | |
| normal | 13 | 4 (30.77%) | |
| amplification | 2 | 1 (50%) | |
| VEGFR1 | 23 | | 0.3401 |
| aneuploid | 1 | 0 (0%) | |
| normal | 18 | 6 (33.33%) | |
| amplification | 4 | 0 (0%) | |

TABLE 2a
Biomarkers Protein Expression

| Biomarker | Analyzed cases | pCR (N, %) | p-value |
|---|---|---|---|
| KISS1 | 25 | | 0.6016 |
| normal | 21 | 7 (33.33%) | |
| overexpressed | 4 | 2 (50%) | |
| VEGFR1 | 22 | | 0.6462 |
| normal | 12 | 4 (33.33%) | |
| overexpressed | 10 | 2 (20%) | |
| HIF | 38 | | 0.3367 |
| normal | 33 | 11 (33.33%) | |
| overexpressed | 5 | 3 (60%) | |
| eNOS | 38 | | 1.000 |
| normal | 34 | 11 (32.5%) | |
| overexpressed | 4 | 1 (25%) | |
| AGTR1 | 26 | | 0.0033 |
| normal | 15 | 1 (6.67%) | |
| overexpressed | 11 | 7 (63.64%) | |

TABLE 2b
AGTR1 Gene Expression

| AGTR1 gene | | | |
|---|---|---|---|
| expression | | | |
| Frequency | | | |
| percent row Pct | Pathological response | | |
| Col Pct | Complete response | non-complete response | Total |
| Normal | 1 | 14 | 15 |
| | 3.85 | 53.85 | 57.69 |
| | 6.67 | 93.33 | |
| | 12.50 | 77.78 | |
| Overexpression | 7 | 4 | 11 |
| | 26.92 | 15.38 | 42.31 |
| | 63.64 | 36.36 | |
| | 87.50 | 22.22 | |
| Total | 8 | 18 | 26 |
| | 30.77 | 69.23 | 100.00 |

Although the number of analyzed cases is rather small, there is a statistical significance association between Angiotensin II type 1 receptor (AGTR1) protein overexpression and pCR patients treated with bevacizumab

TABLE 3a
Biomarkers Gene Expression (referenced to the housekeeping
standard value)

| Biomarker | Analyzed cases | pCR (N, %) | p-value |
|---|---|---|---|
| VEGF-A | 34 | | 0.3235 |
| higher | 1 | 1 (100%) | |
| lower | 33 | 10 (30.3%) | |
| VEGFR1 | 34 | | 0.0693 |
| higher | 7 | 0 (%) | |
| lower | 27 | 11 (40.74%) | |
| HIF | 34 | | 0.4254 |
| higher | 9 | 4 (44.44%) | |
| lower | 25 | 7 (28%) | |
| eNOS | 34 | | 1.000 |
| higher | 1 | 0 (0%) | |
| lower | 33 | 11 (33.33%) | |
| KISS1 | 34 | | 1.000 |
| higher | 1 | 0 (0%) | |
| lower | 33 | 11 (33.33%) | |
| KISS1R | 34 | | 1.000 |
| higher | 0 | — | |
| lower | 34 | 11 (32.35%) | |

TABLE 3b
eNOS Gene Expression

| Pathological | eNOS Gene Expression | | | | |
|---|---|---|---|---|---|
| response | N | Minimum | Maximum | Media | Median |
| Complete | 11 | 0.91 | 3.22 | 2.05 | 2.22 |
| response | | | | | |
| Non-complete | 23 | −1.78 | 3.04 | 1.42 | 1.33 |
| response | | | | | |

Wilcoxon Two-Sample Test

| t Approximation | |
|---|---|
| one-sided Pr > Z | 0.0373 |
| two-sided Pr > \|Z\| | 0.0747 |

Z includes a continuity correction of 0.5

1. A method for identifying a locally advanced, Her2 negative breast cancer patient responsive or sensitive to bevacizumab treatment,
the method comprising determining the expression level of angiotensin II type receptor 1 (AGTR1) in a biological sample of the patient,
identifying the patient as responsive to or sensitive to bevacizumab treatment when an increased expression level of AGTR1 compared to a control level is detected in the sample of the patient, and
administering said bevacizumab treatment to the patient.

2. The method of claim 1, wherein said treatment further comprises chemotherapy.

3. The method of claim 2, wherein said bevacizumab treatment is administered before or after said chemotherapy.

4. The method of claim 2, wherein said chemotherapy comprises docetaxel.

5. The method of claim 2, wherein said chemotherapy comprises doxorubicin and cyclophosphamide.

6. The method of claim 1, wherein said expression level of AGTR1 is determined by detecting protein or mRNA.

7. The method of claim 6, wherein said expression level of AGTR1 is measured by an immunohistochemical (IHC) method or a PCR method.

8. The method of claim 7, wherein said biological sample is a breast tissue biopsy sample or breast tissue resection sample.

9. The method of claim 2, wherein said treatment further comprises one or more anti-cancer therapies.

10. The method of claim 9, wherein said anti-cancer therapy is radiation.

11. The method of claim 1, wherein said biological sample was obtained before neoadjuvant or adjuvant therapy.

12. A method for treating locally advanced, Her2 negative breast cancer, the method comprising administering an effective amount of bevacizumab to a patient identified as responsive or sensitive to bevacizumab therapy, wherein the patient was identified as responsive or sensitive to bevacizumab therapy by detecting an increased expression level of angiotensin II type receptor 1 (AGTR1) in a biological sample from the patient compared to a control level.


SEQUENCE LISTING (14 sequences)

SEQ ID NO: 1 - DNA, 1080 bp, Homo sapiens
atgattctcaactcttctactgaagatggtattaaaagaatccaagatgattgtcccaaagctggaaggcataattacatatttgtcatgattcctactttatacagtatcatctttgtggtgggaatatttggaaacagcttggtggtgatagtcatttacttttatatgaagctgaagactgtggccagtgtttttcttttgaatttagcactggctgacttatgctttttactgactttgccactatgggctgtctacacagctatggaataccgctggccctttggcaattacctatgtaagattgcttcagccagcgtcagtttcaacctgtacgctagtgtgtttctactcacgtgtctcagcattgatcgatacctggctattgttcacccaatgaagtcccgccttcgacgcacaatgcttgtagccaaagtcacctgcatcatcatttggctgctggcaggcttggccagtttgccagctataatccatcgaaatgtatttttcattgagaacaccaatattacagtttgtgctttccattatgagtcccaaaattcaacccttccgatagggctgggcctgaccaaaaatatactgggtttcctgtttccttttctgatcattcttacaagttatactcttatttggaaggccctaaagaaggcttatgaaattcagaagaacaaaccaagaaatgatgatatttttaagataattatggcaattgtgcttttctttttcttttcctggattccccaccaaatattcacttttctggatgtattgattcaactaggcatcatacgtgactgtagaattgcagatattgtggacacggccatgcctatcaccatttgtatagcttattttaacaattgcctgaatcctcttttttatggctttctggggaaaaaatttaaaagatattttctccagcttctaaaatatattcccccaaaagccaaatcccactcaaacctttcaacaaaaatgagcacgctttcctaccgcccctcagataatgtaagctcatccaccaagaagcctgcaccatgttttgaggttgagtga

SEQ ID NO: 2 - protein, 359 aa, Homo sapiens [sequence omitted]

SEQ ID NO: 3 - DNA, 438 bp, Homo sapiens
atgaactcactggtttcttggcagctactgcttttcctctgtgccacccactttggggagccattagaaaaggtggcctctgtggggaattctagacccacaggccagcagctagaatccctgggcctcctggcccccggggagcagagcctgccgtgcaccgagaggaagccagctgctactgccaggctgagccgtcgggggacctcgctgtccccgccccccgagagctccgggagccgccagcagccgggcctgtccgccccccacagccgccagatccccgcaccccagggcgcggtgctggtgcagcgggagaaggacctgccgaactacaactggaactccttcggcctgcgcttcggcaagcgggaggcggcaccagggaaccacggcagaagcgctgggcggggctggggcgcaggtgcggggcagtga

SEQ ID NO: 4 - protein, 137 aa, Homo sapiens
NSLVSWQLLLFLCATHFGEPLEKVASVGNSRPTGQQLESLGLLAPGEQSLPCTERKPAATARLSRRGTSLSPPPESSGSPQQPGLSAPHSRQIPAPQGAVLVQREKDLPNYNWNSFGLRFGKREAAPGNHGRSAGRG

SEQ ID NO: 5 - DNA, 1197 bp, Homo sapiens
atgcacaccgtggctacgtccggacccaacgcgtcctggggggcaccggccaacgcctccggctgcccgggctgtggcgccaacgcctcggacggcccagtcccttcgccgcgggccgtggacgcctggctcgtgccgctcttcttcgcggcgctgatgctgctgggcctggtggggaactcgctggtcatctacgtcatctgccgccacaagccgatgcggaccgtgaccaacttctacatcgccaacctggcggccacggacgtgaccttcctcctgtgctgcgtccccttcacggccctgctgtacccgctgcccggctgggtgctgggcgacttcatgtgcaagttcgtcaactacatccagcaggtctcggtgcaggccacgtgtgccactctgaccgccatgagtgtggaccgctggtacgtgacggtgttcccgttgcgcgccctgcaccgccgcacgccccgcctggcgctggctgtcagcctcagcatctgggtaggctctgcggcggtgtctgcgccggtgctcgccctgcaccgcctgtcacccgggccgcgcgcctactgcagtgaggccttccccagccgcgccctggagcgcgccttcgcactgtacaacctgctggcgctgtacctgctgccgctgctcgccacctgcgcctgctatgcggccatgctgcgccacctgggccgggtcgccgtgcgccccgcgcccgccgatagcgccctgcaggggcaggtgctggcagagcgcgcaggcgccgtgcgggccaaggtctcgcggctggtggcggccgtggtcctgctcttcgccgcctgctggggccccatccagctgttcctggtgctgcaggcgctgggccccgcgggctcctggcacccacgcagctacgccgcctacgcgcttaagacctgggctcactgcatgtcctacagcaactccgcgctgaacccgctgctctacgccttcctgggctcgcacttccgacaggccttccgccgcgtctgcccctgcgcgccgcgccgcccccgccgcccccgccggcccggaccctcggaccccgcagccccacacgcggagctgctccgcctggggtcccacccggcccccgccagggcgcagaagccagggagcagtgggctggccgcgcgcgggctgtgcgtcctgggggaggacaacgcccctctctga

SEQ ID NO: 6 - protein, 398 aa, Homo sapiens
MHTVATSGPNASWGAPANASGCPGCGANASDGPVPSPRAVDAWLVPLFFAALMLLGLVGNSLVIYVICRHKPMRTVTNFYIANLAATDVTFLLCCVPFTALLYPLPGWVLGDFMCKFVNYIQQVSVQATCATLTAMSVDRWYVTVFPLRALHRRTPRLALAVSLSIWVGSAAVSAPVLALHRLSPGPRAYCSEAFPSRALERAFALYNLLALYLLPLLATCACYAAMLRHLGRVAVRPAPADSALQGQVLAERAGAVRAKVSRLVAAVVLLFAACWGPIQLFLVLQALGPAGSWHPRSYAAYALKTWAHCMSYSNSALNPLLYAFLGSHFRQAFRRVCPCAPRRPRRPRRPGPSDPAAPHAELLRLGSHPAPARAQKPGSSGLAARGLCVLGEDNAPL

SEQ ID NO: 7 - DNA, 989 bp, Homo sapiens
agtgtgctggcggcccggcgcgagccggcccggccccggtcgggcctccgaaaccatgaactttctgctgtcttgggtgcattggagcctcgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacaccgcggccgccagcacactg

SEQ ID NO: 8 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 9 - DNA, 4017 bp, Homo sapiens
atggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctctatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagagatcaggaagcaccatacctcctgcgaaacctcagtgatcacacagtggccatcagcagttccaccactttagactgtcatgctaatggtgtccccgagcctcagatcacttggtttaaaaacaaccacaaaatacaacaagagcctggaattattttaggaccaggaagcagcacgctgtttattgaaagagtcacagaagaggatgaaggtgtctatcactgcaaagccaccaaccagaagggctctgtggaaagttcagcatacctcactgttcaaggaacctcggacaagtctaatctggagctgatcactctaacatgcacctgtgtggctgcgactctcttctggctcctattaaccctctttatccgaaaaatgaaaaggtcttcttctgaaataaagactgactacctatcaattataatggacccagatgaagttcctttggatgagcagtgtgagcggctcccttatgatgccagcaagtgggagtttgcccgggagagacttaaactgggcaaatcacttggaagaggggcttttggaaaagtggttcaagcatcagcatttggcattaagaaatcacctacgtgccggactgtggctgtgaaaatgctgaaagagggggccacggccagcgagtacaaagctctgatgactgagctaaaaatcttgacccacattggccaccatctgaacgtggttaacctgctgggagcctgcaccaagcaaggagggcctctgatggtgattgttgaatactgcaaatatggaaatctctccaactacctcaagagcaaacgtgacttattttttctcaacaaggatgcagcactacacatggagcctaagaaagaaaaaatggagccaggcctggaacaaggcaagaaaccaagactagatagcgtcaccagcagcgaaagctttgcgagctccggctttcaggaagataaaagtctgagtgatgttgaggaagaggaggattctgacggtttctacaaggagcccatcactatggaagatctgatttcttacagttttcaagtggccagaggcatggagttcctgtcttccagaaagtgcattcatcgggacctggcagcgagaaacattcttttatctgagaacaacgtggtgaagatttgtgattttggccttgcccgggatatttataagaaccccgattatgtgagaaaaggagatactcgacttcctctgaaatggatggctcctgaatctatctttgacaaaatctacagcaccaagagcgacgtgtggtcttacggagtattgctgtgggaaatcttctccttaggtgggtctccatacccaggagtacaaatggatgaggacttttgcagtcgcctgagggaaggcatgaggatgagagctcctgagtactctactcctgaaatctatcagatcatgctggactgctggcacagagacccaaaagaaaggccaagatttgcagaacttgtggaaaaactaggtgatttgcttcaagcaaatgtacaacaggatggtaaagactacatcccaatcaatgccatactgacaggaaatagtgggtttacatactcaactcctgccttctctgaggacttcttcaaggaaagtatttcagctccgaagtttaattcaggaagctctgatgatgtcagatatgtaaatgctttcaagttcatgagcctggaaagaatcaaaacctttgaagaacttttaccgaatgccacctccatgtttgatgactaccagggcgacagcagcactctgttggcctctcccatgctgaagcgcttcacctggactgacagcaaacccaaggcctcgctcaagattgacttgagagtaaccagtaaaagtaaggagtcggggctgtctgatgtcagcaggcccagtttctgccattccagctgtgggcacgtcagcgaaggcaagcgcaggttcacctacgaccacgctgagctggaaaggaaaatcgcgtgctgctccccgcccccagactacaactcggtggtcctgtactccaccccacccatctag

SEQ ID NO: 10 - protein, 1338 aa, Homo sapiens [sequence omitted]

SEQ ID NO: 11 - DNA, 2481 bp, Homo sapiens
atggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactag

SEQ ID NO: 12 - protein, 825 aa, Homo sapiens
EGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 13 - DNA, 3612 bp, Homo sapiens
atgggcaacttgaagagcgtggcccaggagcctgggccaccctgcggcctggggctggggctgggccttgggctgtgcggcaagcagggcccagccaccccggcccctgagcccagccgggccccagcatccctactcccaccagcgccagaacacagccccccgagctccccgctaacccagcccccagaggggcccaagttccctcgtgtgaagaactgggaggtggggagcatcacctatgacaccctcagcgcccaggcgcagcaggatgggccctgcaccccaagacgctgcctgggctccctggtatttccacggaaactacagggccggccctcccccggccccccggcccctgagcagctgctgagtcaggcccgggacttcatcaaccagtactacagctccattaagaggagcggctcccaggcccacgaacagcggcttcaagaggtggaagccgaggtggcagccacaggcacctaccagcttagggagagcgagctggtgttcggggctaagcaggcctggcgcaacgctccccgctgcgtgggccggatccagtgggggaagctgcaggtgttcgatgcccgggactgcaggtctgcacaggaaatgttcacctacatctgcaaccacatcaagtatgccaccaaccggggcaaccttcgctcggccatcacagtgttcccgcagcgctgccctggccgaggagacttccgaatctggaacagccagctggtgcgctacgcgggctaccggcagcaggacggctctgtgcggggggacccagccaacgtggagatcaccgagctctgcattcagcacggctggaccccaggaaacggtcgcttcgacgtgctgcccctgctgctgcaggccccagatgagcccccagaactcttccttctgccccccgagctggtccttgaggtgcccctggagcaccccacgctggagtggtttgcagccctgggcctgcgctggtacgccctcccggcagtgtccaacatgctgctggaaattgggggcctggagttccccgcagcccccttcagtggctggtacatgagcactgagatcggcacgaggaacctgtgtgaccctcaccgctacaacatcctggaggatgtggctgtctgcatggacctggatacccggaccacctcgtccctgtggaaagacaaggcagcagtggaaatcaacgtggccgtgctgcacagttaccagctagccaaagtcaccatcgtggaccaccacgccgccacggcctctttcatgaagcacctggagaatgagcagaaggccagggggggctgccctgcagactgggcctggatcgtgccccccatctcgggcagcctcactcctgttttccatcaggagatggtcaactatttcctgtccccggccttccgctaccagccagacccctggaaggggagtgccgccaagggcaccggcatcaccaggaagaagacctttaaagaagtggccaacgccgtgaagatctccgcctcgctcatgggcacggtgatggcgaagcgagtgaaggcgacaatcctgtatggctccgagaccggccgggcccagagctacgcacagcagctggggagactcttccggaaggcttttgatccccgggtcctgtgtatggatgagtatgacgtggtgtccctcgaacacgagacgctggtgctggtggtaaccagcacatttgggaatggggatcccccggagaatggagagagctttgcagctgccctgatggagatgtccggcccctacaacagctcccctcggccggaacagcacaagagttataagatccgcttcaacagcatctcctgctcagacccactggtgtcctcttggcggcggaagaggaaggagtccagtaacacagacagtgcaggggccctgggcaccctcaggttctgtgtgttcgggctcggctcccgggcatacccccacttctgcgcctttgctcgtgccgtggacacacggctggaggaactgggcggggagcggctgctgcagctgggccagggcgacgagctgtgcggccaggaggaggccttccgaggctgggcccaggctgccttccaggccgcctgtgagaccttctgtgtgggagaggatgccaaggccgccgcccgagacatcttcagccccaaacggagctggaagcgccagaggtaccggctgagcgcccaggccgagggcctgcagttgctgccaggtctgatccacgtgcacaggcggaagatgttccaggctacaatccgctcagtggaaaacctgcaaagcagcaagtccacgagggccaccatcctggtgcgcctggacaccggaggccaggaggggctgcagtaccagccgggggaccacataggtgtctgcccgcccaaccggcccggccttgtggaggcgctgctgagccgcgtggaggacccgccggcgcccactgagcccgtggcagtagagcagctggagaagggcagccctggtggccctccccccggctgggtgcgggacccccggctgcccccgtgcacgctgcgccaggctctcaccttcttcctggacatcacctccccacccagccctcagctcttgcggctgctcagcaccttggcagaagagcccagggaacagcaggagctggaggccctcagccaggatccccgacgctacgaggagtggaagtggttccgctgccccacgctgctggaggtgctggagcagttcccgtcggtggcgctgcctgccccactgctcctcacccagctgcctctgctccagccccggtactactcagtcagctcggcacccagcacccacccaggagagatccacctcactgtagctgtgctggcatacaggactcaggatgggctgggccccctgcactatggagtctgctccacgtggctaagccagctcaagcccggagaccctgtgccctgcttcatccggggggctccctccttccggctgccacccgatcccagcttgccctgcatcctggtgggtccaggcactggcattgcccccttccggggattctggcaggagcggctgcatgacattgagagcaaagggctgcagcccactcccatgactttggtgttcggctgccgatgctcccaacttgaccatctctaccgcgacgaggtgcagaacgcccagcagcgcggggtgtttggccgagtcctcaccgccttctcccgggaacctgacaaccccaagacctacgtgcaggacatcctgaggacggagctggctgcggaggtgcaccgcgtgctgtgcctcgagcggggccacatgtttgtctgcggcgatgttaccatggcaaccaacgtcctgcagaccgtgcagcgcatcctggcgacggagggcgacatggagctggacgaggccggcgacgtcatcggcgtgctgcgggatcagcaacgctaccacgaagacattttcgggctcacgctgcgcacccaggaggtgacaagccgcatacgcacccagagcttttccttgcaggagcgtcagttgcggggcgcagtgccctgggcgttcgaccctcccggctcagacaccaacagcccctga

SEQ ID NO: 14 - protein, 1203 aa, Homo sapiens
MGNLKSVAQEPGPPCGLGLGLGLGLCGKQGPATPAPEPSRAPASLLPPAPEHSPPSSPLTQPPEGPKFPRVKNWEVGSITYDTLSAQAQQDGPCTPRRCLGSLVFPRKLQGRPSPGPPAPEQLLSQARDFINQYYSSIKRSGSQAHEQRLQEVEAEVAATGTYQLRESELVFGAKQAWRNAPRCVGRIQWGKLQVFDARDCRSAQEMFTYICNHIKYATNRGNLRSAITVFPQRCPGRGDFRIWNSQLVRYAGYRQQDGSVRGDPANVEITELCIQHGWTPGNGRFDVLPLLLQAPDEPPELFLLPPELVLEVPLEHPTLEWFAALGLRWYALPAVSNMLLEIGGLEFPAAPFSGWYMSTEIGTRNLCDPHRYNILEDVAVCMDLDTRTTSSLWKDKAAVEINVAVLHSYQLAKVTIVDHHAATASFMKHLENEQKARGGCPADWAWIVPPISGSLTPVFHQEMVNYFLSPAFRYQPDPWKGSAAKGTGITRKKTFKEVANAVKISASLMGTVMAKRVKATILYGSETGRAQSYAQQLGRLFRKAFDPRVLCMDEYDVVSLEHETLVLVVTSTFGNGDPPENGESFAAALMEMSGPYNSSPRPEQHKSYKIRFNSISCSDPLVSSWRRKRKESSNTDSAGALGTLRFCVFGLGSRAYPHFCAFARAVDTRLEELGGERLLQLGQGDELCGQEEAFRGWAQAAFQAACETFCVGEDAKAAARDIFSPKRSWKRQRYRLSAQAEGLQLLPGLIHVHRRKMFQATIRSVENLQSSKSTRATILVRLDTGGQEGLQYQPGDHIGVCPPNRPGLVEALLSRVEDPPAPTEPVAVEQLEKGSPGGPPPGWVRDPRLPPCTLRQALTFFLDITSPPSPQLLRLLSTLAEEPREQQELEALSQDPRRYEEWKWFRCPTLLEVLEQFPSVALPAPLLLTQLPLLQPRYYSVSSAPSTHPGEIHLTVAVLAYRTQDGLGPLHYGVCSTWLSQLKPGDPVPCFIRGAPSFRLPPDPSLPCILVGPGTGIAPFRGFWQERLHDIESKGLQPTPMTLVFGCRCSQLDHLYRDEVQNAQQRGVFGRVLTAFSREPDNPKTYVQDILRTELAAEVHRVLCLERGHMFVCGDVTMATNVLQTVQRILATEGDMELDEAGDVIGVLRDQQRYHEDIFGLTLRTQEVTSRIRTQSFSLQERQLRGAVPWAFDPPGSDTNSP